(19) United States
(12) Patent Application Publication
(10) Pub. No.: US 2007/0190646 A1
(43) Pub. Date: Aug. 16, 2007

(54) Regulating stem cell differentiation by controlling matrix elasticity

(75) Inventors: Adam J. Engler, Philadelphia, PA (US); H. Lee Sweeney, Philadelphia, PA (US); Dennis Discher, Philadelphia, PA (US)

Correspondence Address: MONTGOMERY, MCCRACKEN, WALKER & RHOADS, LLP, 123 SOUTH BROAD STREET, AVENUE OF THE ARTS, PHILADELPHIA, PA, 19109, US

(73) Assignee: The Trustees of the University of Pennsylvania

(21) Appl. No.: 11/351,420

(22) Filed: Feb. 10, 2006

(51) Int. Cl. C12N 5/02; C12N 5/00

(52) U.S. Cl. 435/325

(57) ABSTRACT

Provided are methods for the selection and regulation of the mechanical properties of substrates or tissue microenvironments as a technique to regulate in vitro differentiation, cell shape and/or lineage commitment of anchorage-dependent cells, such as mesenchymal stem cells into, e.g., neurogenic-, myogenic-, and osteogenic-type cells. Substrate mechanical properties include elasticity, tension, adhesion, and myosin-based contractile mechanisms. Inhibitors can be introduced to further regulate differentiation.

### GOVERNMENT SUPPORT

This invention was supported in part by funds obtained from the National Institutes of Health: “Myocytes Sense Substrate Stiffness—New materials,” grant number 11R21EB004489-01 and “Bioengineering Research Partnership—Muscular Dystrophy,” grant number 5R01AR047292-05. The U.S. government may therefore have certain rights in the invention.

### FIELD OF THE DISCLOSURE

The present disclosure relates to differentiating cells based on mechanical properties of the environment of the cell, in particular, elasticity of the surrounding matrix.

### BACKGROUND

Normal tissue cells are generally not viable when suspended in a fluid. Thus, they are “anchorage-dependent” because to grow, such cells must adhere to a solid matrix, varying in stiffness from rigid glass to soft agar, topography, and thickness (e.g., basement membrane). Anchorage-dependent cells, therefore, are no longer viable if dissociated from the solid matrix and suspended in the culture media, even if soluble proteins are added to engage cell adhesion molecules, e.g., integrin-binding RGD peptide.

Fluids are clearly mechanically distinct from solids, which flow when stressed, whereas solids have the ability to resist sustained deformation. In most soft tissues—skin, muscle, brain, etc.—adherent cells together with an extracellular matrix constitute a relatively elastic microenvironment. Macroscopically, elasticity (measured as ‘Pascal’ or newtons/square meters) is evident in the ability of a solid tissue to recover its shape within seconds after mild poking and pinching, or even after sustained compression. At the cellular scale, normal tissue cells probe elasticity as they adhere and pull on their surroundings. Such processes are dependent in part on myosin-based contractility and transcellular adhesions—centered on integrins, cadherins, and perhaps other adhesion molecules—to transmit forces to substrates. Consequently, adhesion complexes and the actomyosin cytoskeleton, whose contractile forces are transmitted through transcellular structures, play key roles in molecular pathways.

Microenvironments and niches appear important in stem cell lineage specification and differentiation as cells can ‘feel’ tissue softness via contractile forces, generated by cross-bridging interactions of actin and myosin filaments. These forces (referred to as traction forces) are transmitted to the substrate, causing wrinkles or strains in thin films or soft gels (Harris et al., Science 208:177 (1980); Oliver et al., J. Cell Biol. 145:589 (1999); Marganski et al., Methods Enzymol. 361:197 (2003); Balaban et al., Nat. Cell Biol. 3:466 (2001); Tan et al., Proc. Natl. Acad. Sci. USA 100:1484 (2003)). The cell, in turn, responds to the resistance of the substrate by adjusting its adhesions, cytoskeleton, and overall state, e.g.—differentiation. Although considerable attention has been directed at the responsiveness of individual differentiated cells to external forces (outside-in) such as stretching and local twisting (Alenghat et al., Sci. STKE 119:pe6 (2002)), there is little understanding of how cell-exerted forces in response to the surrounding microenvironment contribute to signaling pathways effecting contractile mechanisms and ultimately cell state.

For example, adult stem cells, as part of normal regenerative processes, are believed to migrate or circulate and engraft to sites of injury, and will differentiate within these various in vivo microenvironments, ranging from compliant tissue substrates, such as brain or muscle, to rigid tissue substrates, such as bone. Mesenchymal stem cells (MSCs) are pluripotent, anchorage-dependent, and bone marrow-derived cells differentiating into various types of anchorage-dependent cells, including neurons, myoblasts, osteoblasts, and more (Gang et al., Stem Cells 22:617-624 (2004); Gilbert et al., J. Biol. Chem. 277, 2695-2701 (2002); McBeath et al., Developmental Cell 6: 483-495 (2004); Pittenger et al., Science 284:143-147 (1999); Salim et al., J. Biol. Chem. 279:40007-40016 (2004); Tanaka et al., J. Cell Biochem. 93, 454-462 (2004)) via different signaling paths. Soluble factors and cell density clearly influence these differentiation pathways chemically, but variations can also be physical (Gregory et al., Science STKE PE37 (2005); Salasznyk et al., J. Biomed. Biotechnol. 24-34 (2004)). For instance, stem cells adhere and differentiate in soft brain tissue or near rigid bone, and in vitro on soft gels or hard plastic culture dishes. However, compounding MSC-based therapies which consider physical matrix effects are normal wound healing responses, where the formation of fibrotic scar tissue will stiffen the microenvironment, and genetic disorders, such as muscular dystrophy, which increase fibrosis in affected tissues (Engler et al., 2004c, supra).

This wide range in substrate stiffness, exacerbated by disease, has been observed in vivo in many differentiated cell types to strongly influence focal adhesions and cytoskeleton (Beningo et al., J. Cell Biol. 153:881-888 (2001); Bershadshy et al., Annu. Rev. Cell Dev. Biol. 19:677-695 (2003); Discher et al., Science, 310:1139-1143 (Nov. 18, 2005); Engler et al., Biophys. J. 86:617-628 (2004a); Engler et al., J. Cell Biol. 166: 877-887 (2004c); Georges et al., J. Appl. Physiol. 98:1547-1553 (2005); Pelham et al., Proc. Natl. Acad. Sci. USA 94:13661-13665 (1997); Yeung et al., Cell Motil. Cytoskeleton 60:24-34 (2005)) and to be modulated by Ras superfamily proteins and their effectors (Gregory et al., 2005, supra; Paszek et al., Cancer Cell 8:241-254 (2005); Peyton et al., J. Cell Physiol. 204:198-209 (2005)). Rho subfamily members especially are broadly known to regulate the cytoskeleton, cell growth, and transcription, and recent studies of stem cell differentiation are also beginning to implicate cytoskeletal reorganization in vitro (Rodrigues et al., J. Cell Biochem. 93:721-731 (2004)) and Ras superfamily signaling in vivo (Benitah et al., Science 309:933-935 (2005)).

In fibroblasts, it is well established that Rho-stimulated contractility drives stress fiber and focal adhesion formation and that smooth muscle actin up-regulation correlates with contractility on rigid substrates (Chrzanowska-Wodnicka et al., J. Cell Biol. 133:1403 (1996); Hinz et al., Mol. Biol. Cell 12:2730 (2001)). Rac1, another Rho family protein, in activated macrophages promotes engulfment of soft beads, which otherwise are not engulfed (Beningo et al., 2001, supra). RhoA, in contrast, has no observable effect in these measurements. Current views of signaling pathways, especially various physical signals, clearly implicate Rac in cell motility (versus contractility) -indeed, myosin inhibition activates Rac (Katsumi et al., J. Cell Biol. 158:153 (2002)).

In addition to cell differentiation, the mechanical resistance or elasticity of a tissue cell's surrounding microenvironment adjusts spread morphology and contractile forces (Cukierman et al., Science 294:1708-1712 (2001); Engler et al., 2004a, supra; Flanagan et al., Neuroreport 13:2411-2415 (2002); Tolic-Norrelykke et al., Am. J Physiol. Cell Physiol. 283:C1254-1266 (2002)), as well as motility and viability (Engler et al., 2004c, supra; Lo et al., Biophys. J. 79:144-152 (2000); Peyton et al., 2005, supra; Wang et al., Am. J. Physiol. Cell Physiol. 279:C1345-1350 (2000); Wong et al., Langmuir 19:1908-1913 (2003)), and protein expression and signaling (Beningo et al., 2001, supra; Pelham et al., 1997, supra). The involvement of contractile-effector proteins in sensing implies that cell crawling, and thus MSC's ability migrate or circulate and engraft to sites of injury is also likely to be sensitive to substrate stiffness, as demonstrated in studies of the “cell on gel” effect with epithelial cells (Pelham et al., 1997, supra), fibroblasts (Lo et al., 2000, supra), and smooth muscle cells (Peyton et al., 2005, supra; Zaari et al., Adv. Mater. 16:2133 (2004)). With the latter cell type, crawling speed appears maximal at an intermediate stiffness and is reminiscent of crawling speed versus adhesive ligand concentration (Goodman et al., J. Cell Biol. 109:799 (1989))—mathematically modeled as a shift in the balance between ligand-mediated traction and ligand-mediated anchorage (Zaman et al., Biophys. J. 89:1389 (2005)). Additionally, smooth muscle cells on gels are slowed by inhibition of Rho kinase, suggesting that RhoA activity contributes to the tensions needed to detach any established adhesions at the rear of a motile cell (a process not needed in engulfinent) (Jay et al., J. Cell Sci. 108:387 (1995)). The dependence of cell crawling speed and direction on substrate stiffness, particularly gradients in stiffness, is referred to as “durotaxis” (Lo et al., 2000, supra).

Nevertheless, while cells have been shown to respond to externally applied forces (see, e.g., Riveline et al., J. Cell Biol. 153:1175-1186 (2001)), until the present invention there was no suggestion of a relationship between pluripotent cell differentiation and matrix elasticity and how various disease states can complicate the physical remodeling required to decrease elasticity to proper, tissue-relevant levels prior to the use of stem-cell based therapies. Thus a need remained in the art to provide a method for regulating the differentiation of mesenchymal stem cells (“MSCs”) into anchorage-dependent cell types. Moreover, similar sensitivity, growth and remodeling principles seem to apply to most anchored cells, and by regulating differentiation via contractile mechanisms, light may be shed on other matrix-altering pathologies.

### SUMMARY OF THE INVENTION

A normal tissue cell not only applies forces, but also, as demonstrated in the following disclosure, responds through cytoskeleton organization (and other cellular processes) to the resistance that the cell senses, regardless of whether the resistance derives from a normal tissue matrix, synthetic substrate, or even an adjacent cell. Thus, the present invention meets the foregoing identified needs and other purposes by providing methods for regulating differentiation and cell shape of an anchorage-dependent cell.

It is, therefore, an object of the present invention to provide a method for regulating differentiation and cell shape of an anchorage-dependent cell, comprising: selecting, designing, or engineering a substrate or tissue microenvironment having an elasticity defined by elastic constant E; introducing the anchorage-dependent cell onto a substrate or into a microenvironment; and developing the anchorage-dependent cell into a differentiated cell type, wherein shape and lineage commitment (in terms of gene or protein expression, or both) are regulated by the elasticity of the underlying substrate. Depending on the controlled elasticity of the substrate, there is implemented a differentiation of the anchorage-dependent cells into at least one neurogenic, myogenic or osteogenic-type cell.

It is an additional object of the invention to provide a method wherein the subject anchorage-dependent cell is exposed to an inhibiting agent to inhibit expression of a lineage-specific regulator.

It is a further object of the invention to provide a method for regulating differentiation and cell shape of an anchorage-dependent cell, comprising: selecting, designing, or engineering a substrate or tissue microenvironment having an elasticity defined by elastic constant E; and introducing the anchorage-dependent cell onto a substrate or into a microenvironment, as above; and balancing chemo-mechanical energetics localized to cell adhesions against contractile energetics, σ, of the cell that balances cell traction stresses, τ, exerted by the cell on its underlying substrate, thereby controlling cell shape and lineage commitment. Because the cell adhesions provide necessary attachments permitting the cell to feel its microenvironment, and adhesion area increases linearly with E, larger deformation within the cell occurs on stiffer matrices and larger deformation in the substrate occurs on softer matrices.

It is yet another object of the invention to provide a method wherein the cell shape and differentiation of the subject anchorage-dependent cell is further regulated by controlling cell strain, such that there is an inverse relationship between intracellular and extracellular strains so that on stiff matrices, cell strains are large, while matrix strains are small, and on soft matrices, cell strains are small, while matrix strains are large.

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended drawings in which like numerals designate like elements. It should be understood, however, that the invention is not limited to the precise arrangements and instrumentalities shown.

FIGS. 1A-1C show results for mesenchymal stem cell differentiation as a result of tissue elasticity. FIG. 1A graphically shows that solid tissues exhibit a range of elastic moduli, E. FIG. 1B schematically illustrates a mesenchymal stem cell (MSC) and the environment used in the in vitro gel system of the present invention, allowing for a tunable elastic modulus through changes in cross-link density, for control of cell adhesion by covalent attachment of collagen-I, and for control of thickness h by adjusting the volume of polymerizing solution. Also shown in FIG. 1B presents MSC images corresponding to different substrate elasticity (0.1-1 kPa, 8-17 kPa and 30-40 kPa) and different times (4 and 96 hours). The scale bar is 20 μm. Graphs inset in FIG. 1B quantify morphological changes versus E. Graph (i) shows cell branching per length of primary E13.5 mouse neurons, MSCs, and blebbistatin-treated MSCs and graph (ii) shows spindle morphology of MSCs, blebbistatin-treated MSCs, and mitomycin C-treated MSCs (open squares) compared to C2C12 myoblasts (dashed line). FIG. 1C shows microarray profiling results of MSC transcripts in cells cultured on 0.1, 1, 11, and 34 kPa matrices displayed as a relative change (+ or −) from initially isolated MSCs. Neurogenic markers (left) are highest on 0.1-1 kPa gels, myogenic markers (center) are highest on 11 kPa gels, and osteogenic markers (right) are highest on 34 kPa gels.

FIGS. 2A-2C show protein and transcript profiles as a function of elasticity for MSCs. FIG. 2A shows the neuronal cytoskeletal marker, P-NFH, expressed in neurite-like extensions (arrows) of MSCs (>75%) only on soft, neurogenic gels (E_{brain}≈0.1-1 kPa). The muscle transcription factor MyoD1 is up-regulated and nuclear-localized (arrow) only in MSCs on myogenic gels (E_{muscle}≈8-17 kPa). Likewise, the osteoblast transcription factor CBFα1 (arrow) is only expressed on stiff, osteogenic gels (Eₒₛₜₑₒ≈30-40 kPa). In FIG. 2A the scale bar is 5 μm. FIG. 2B shows microarray profiles of MSCs cultured on E_{muscle} and Eₒₛₜₑₒ gels (of 11 and 34 kPa), normalized to profiles of C2C12-myoblasts and hFOB-osteoblasts. FIG. 2C graphs the fluorescent intensity of differentiation markers in cells versus substrate elasticity and reveals a maximum lineage commitment at the E value typical of each tissue type. Fits are to Equation 3, below. Average intensity is normalized to peak expression of control cells (C2C12 or HFOB), for which only the fits are shown.

FIGS. 3A-3D graphically show lineage commitment as stimulated by a combination of substrate elasticity and soluble factors for the cell types defined in the figure legends. In FIG. 3A, expression of NFH and P-NFH in MSC neurite-like extensions is highest for substrates with E<1 kPa. Inset images show growth cone-like, NFH-containing structures (arrows) on soft, but not on moderately stiff substrates. β-tubulin western blotting shows expression on soft gels only. FIGS. 3B and 3C, show fluorescent intensities of MyoD1 and CBFα1 in MSCs peak near E_{muscle} (8-17 kPa) and Eₒₛₜₑₒ(>30 kPa), respectively. In FIG. 3D, western blots confirm lineage commitment with matrix or soluble ligand alone. When normalized to actin, CBFα1 and MyoD expression only reached control levels when both matrix elasticity and soluble ligand are conducive for differentiation.

FIG. 4 shows that myogenic lineage commitment stops short of skeletal muscle protein expression. Skeletal muscle myosin heavy chain-stained MSCs showed minimal expression after 7 days, regardless of matrix stiffness or stimulus; whereas by comparison, myoblasts show 5- to 10-fold higher expression regardless of the matrix mechanics.

FIGS. 5A-5C show that myosin-based contractility induces differentiation and influences organization of MSCs. In FIG. 5A, expression of a range of myosin genes shows graded sensitivity to stiffness (Var), and an overall average expression (Avg) that is up-regulated on stiffer matrices. In FIG. 5B, immunoblots for non-muscle myosins (NMM IIA and B) show the greatest variation for NMM IIB. Both myosins and the various markers for differentiation showed stiffness sensitivity, as well as inhibition by blebbistatin-neurogenic (β3-tubulin), myogenic (MyoD and Desmin), and osteogenic (CBFα1). In FIG. 5C, immunofluorescence of NMM IIB, normalized to uncommitted MSCs, showed similar stiffness sensitivity that did not change with induction media (i.e., MIM, or OIM). Inset images of NMM II indicate similar expression, but show distinct, blebbistatin-inhibitable organization of NMM II, with striations (arrowheads) on E_{muscle}-gels (11 kPa) and stress fibers on the stiffer substrates (34 kPa). The scale bar is 5 μm.

FIGS. 6A-4E show that Rho-GTPase signaling correlates with lineage commitment and adhesion. In FIG. 6A, activator/effector transcripts (top) are generally stiffness-dependent, with high variance (Var). Adhesion and cortical linker transcripts (bottom) typically show less variation, but a larger average (Avg) increase with matrix stiffness. FIG. 6B shows cross-correlation of activators/effectors with lineage-specific transcription factors (STAT3 for neurogenic; MyoD for myogenic; and CBFα1 for osteogenic). FIG. 6C shows that inhibition of the Rho-GTPase, ROCK, by Y27632 does not influence MyoD expression on myogenic gels (11 kPa), based on immunoblots, but it does block CBFα1 expression on osteogenic gels (34 kPa). Also, C2C12 myoblast expression of MyoD was not affected by ROCK inhibition, whereas hFOB osteoblast expression of CBFα1 was inhibited. FIG. 6D presents a series of images showing that paxillin-labeled MSC adhesions grow from almost undetectable diffuse ‘contacts’ on neurogenic, soft gels (1 kPa) to numerous punctate adhesions on stiffer, myogenic gels (11 kPa). Finally, in FIGS. 6E and 6F-actin organization shows a similar trend—diffuse on soft gels to progressively organized on stiff gels and on glass (as stress fibers). The scale bar is 20 μm.

FIGS. 7A-7B show that matrix stiffness alters the adhesion-contractile balance. FIG. 7A shows that focal adhesion size as a percentage of cell area increases with matrix elasticity. The inset shows focal adhesion distributions scale with focal adhesion size, and shift to larger adhesions for higher stiffness substrates. FIG. 7B shows contractility or cell pre-stress, σ (the net tensile force over the cell-matrix interface carried by the actin cytoskeleton across a cross-sectional area of the cell that balances cell traction forces), in both MSCs and control cell lines increases linearly with substrate elasticity, E. The inset image shows a myoblast (outlined) displacing beads embedded in the gel (black arrows) that equates to a strain field represented by the color map (white is high strain). The scale bar is 10 μm. In the lower plot of FIG. 7B, membrane cortical stiffness (measured by micropipette aspiration) increases with gel stiffness, but blebbistatin treatment softens all cell membranes more than 3-fold. The middle inset of FIG. 7B shows mean intracellular strain, εᵢₙ, versus the mean extracellular strain, εₒᵤₜ, fit to a power-law (εᵢₙ=B*εₒᵤₜ^{b}) for all cell types.

FIG. 8 shows cell spreading differences between cells grown on an ultra-thin polyacrylamide gel, <500 nm, and a polyacrylamide gel of normal thickness, i.e. −70 microns. As shown, cell spreading on thin gels can be mapped to that of thick gels to determine an ‘apparent’ gel modulus for cells on these thinner materials (inset graph). Dark gray shaded region represents Eₐₚₚₐᵣₑₙₜ, given experimental uncertainties.

FIGS. 9A-9B show how the cell membrane's cortical stiffness in FIG. 7B was measured. FIG. 9A shows the micropipette schematic for membrane aspiration. FIG. 9B shows sample aspiration data of 5 cells from a single matrix stiffness fit by Equation 3, showing the range of variability. The inset images illustrate this phenomenon, with arrows indicating the membrane cap. The scale bar is 5 μm.

FIG. 10 summarizes the observed elasticity-coupled lineage commitment for a pluripotent mesenchymal stem cell.

### DETAILED DESCRIPTION OF CERTAIN PREFERRED EMBODIMENTS

The present invention provides a method for regulating the differentiation of mesenchymal stem cells (“MSCs”) in response to tissue elasticity, with coupled regulation of non-muscle myosin II (“NMM II”) activity, as well as cytoskeletal organization. In some aspects of the invention, cell morphology shows that lineage commitment is influenced by matrix stiffness (elasticity), and that it is dependent on NMM II (particularly isoforms IIA, IIB, and IIC).

Regardless of geometry, the intrinsic resistance of a solid to a stress is measured by the solid's elastic (or Young's) modulus E, which is most simply obtained by applying a force—such as hanging a weight—to a section of tissue or other material and then measuring the relative change in length or strain. Another common method to obtain E involves controlled macro- or micro-indentation, including atomic force microscopy (AFM). The elastic modulus E is discussed, e.g., by Sugawara et al., Hearing Research 192:57-64 (2004); Taylor et al. J. Biomech. 37:1263-1269 (2004); Engler et al., 2004c, supra. Many tissues and biomaterials exhibit a relatively linear stress versus strain relation up to small strains of about 10 to 20%. The slope E of stress versus strain is relatively constant at the small strains exerted by cells (Lo et al., 2000, supra), although stiffening (increased E) at higher strains is the norm (Storm et al., Nature 435:191 (2005); Fung, A First Course in Continuum Mechanics: For Physical and Biological Engineers and Scientists (Prentice Hall, Englewood Cliffs, N.J., ed. 3, 1994).

Nonetheless, microscopic views of both natural and synthetic matrices (e.g., collagen fibrils and polymer-based mimetics (Stevens et al., Science 310:1135 (2005)), suggest that there are many subtleties to tissue mechanics, particularly concerning the length and time scales of greatest relevance to cell sensing. The elastic resistance that a cell ‘feels’ when it attaches to a substrate is governed by the elastic constant E of the substrate or tissue microenvironment. Sample preparation is also critical; for example, macroscopic elastic moduli measurements of whole brain can vary 2-fold or more, depending on sample preparation, perfusion, etc. (Gefen et al., J. Biomech. 37:1339 (2004)). In addition, many single or multi-cell probing methods involve high-frequency stressing (Hu et al., Am. J. Physioi. Cell Physiol. 287:C1184 (2004)), whereas relevant time scales for cell-exerted strains seem likely to range from seconds to hours, motivating long time studies of cell rheology (Bao et al., Nat. Mater. 2:715 (2003); Wottawah etal., Phys. Rev. Lett. 94:098103 (2005)). Regardless, comparisons of E (in units of Pascal; “Pa”) of three diverse tissues that contain a number of different cell types show that brain tissue is softer than muscle (skeletal muscle) (Engler et al., 2004c, supra; Yoshikawa et al., Biochem. Biophys. Res. Commun. 256:13 (1999)), and muscle is softer than collagenous bone (Engler et al., 2004c, supra; Taylor et al., J. Biomech. 37:1263-1269 (2004)). Although mapping soft tissue micro-elasticities at a resolution typical in histology is important, the implication here is that there are distinct elastic microenvironments for epithelial cells and fibroblasts in skin, for myotubes in fiber bundles, for neurons in brain, etc.

Correlations have long been made between increased cell adhesion and increased cell contractility (e.g., Leader et al., J. Cell Sci. 64:1 (1983)), but it now seems clear that tactile sensing of substrate stiffness feeds back on adhesion and cytoskeleton, as well as on net contractile forces, for many cell types. Seminal studies on epithelial cells and fibroblasts exploited inert polyacrylamide gels with a thin coating of covalently attached collagen (Pelham et al., 1997, supra). This adhesive ligand allows the cells to attach and, by controlling the extent of polymer cross-linking in the gels, E can be adjusted over several orders of magnitude, from extremely soft to stiff.

Because tissue elasticity is a factor in MSC differentiation, matrix elasticity is mimicked in vitro in the present invention with relatively inert, collagen I-coated polyacrylamide gels in which the concentration of bis-acrylamide cross-linking sets the elasticity (Pelham et al., 1997, supra). Solid phase gels for 2-dimensional electrophoresis generally are made of a porous polymer, such as polyacrylamide, and are constructed using known methods.

To minimize variability, it is beneficial if the materials and methods for making the gels are reproducible (see, e.g., the Examples that follow), and perhaps, produced by an automated means to reduce introduced variability. Gel monomers are mixed with agents that induce polymerization and then are poured into a mold that dictates the size and shape of the polymerized gel. For example, the catalyzed liquid gel monomer can be poured between glass plates separated uniformly over the entire surfaces thereof to produce a square or rectangular slab gel. The glass plates, separated by about a millimeter or a fraction thereof, are held in place until the gel is formed. The concentrations of polyacrylamide gels used in electrophoresis are generally stated in terms of % T (the total percentage of acrylamide in the gel by weight) and % C (the proportion of the total acrylamide that is accounted for by the cross-linker used). N,N′-methylenebisacrylamide (“bis”) is typically used as a cross-linker.

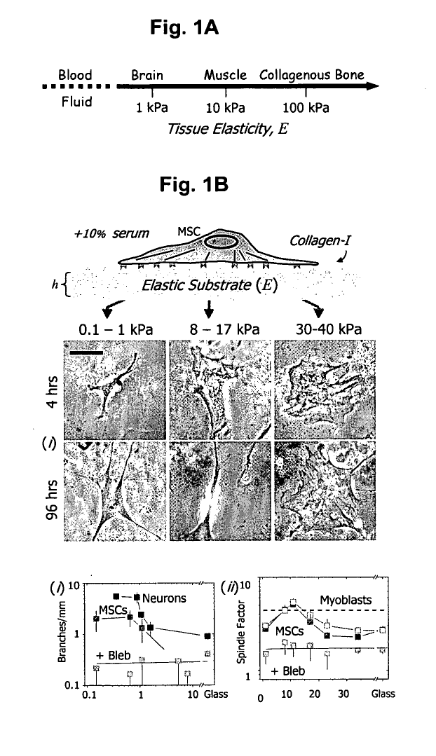

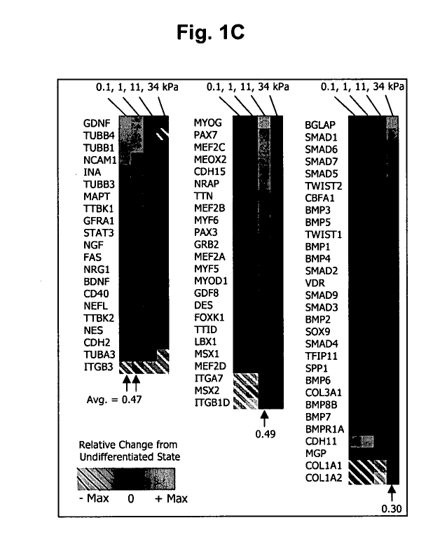

Using these tunable gel systems and sparse cultures, FIGS. 1A-1C show exemplary results for MSC differentiation as a result of tissue elasticity. For preparation, see e.g., the Examples that follow. A stable population of MSCs was biased towards developing different lineages, i.e., neurons, myoblasts, and osteoblasts—all in identical serum conditions and all on collagen-I (see, e.g., FIG. 1B), thereby supporting differentiation into these three phenotypes (Engler et al., 2004c, supra; Garcia et al., J. Dent. Res. 84:407-413 (2005); Stephansson et al., Biomaterials 23:2527-2534 (2002); Yoneno et al., J. Biomed. Mater. Res. A, in press (2005)). In support of differentiation, the outside-in, E-regulation of key lineage markers and myosins, as well as activators/effectors, such as Rho that cross-correlates with lineage-specific transcription factors and metrics of contractility, were monitored and documented (see Examples).

In an alternative embodiment of the present invention, MSC differentiation is blocked or inhibited with an inhibitor of NMM II, blebbistatin. Nevertheless, as demonstrated herein in the Examples that follow, soluble inductive factors tend to be less selective than matrix stiffness in stimulating differentiation. Moreover, by controlling gel thickness h, the distance between a MSC and its substrate (that influences differentiation) was determined, and physically defined the microenvironment surrounding the MSCs.

As graphically shown in FIG. 1A, solid tissues exhibit a range of elasticity, as measured by the elastic modulus, E (the ratio of stress to strain, providing a measure of the stiffness of a material), ranging from less than 1 kPa to more than 100 kPa, depending on cell type. For example, E values of about 1 kPa generally correspond to brain tissue, E values about 10 kPa generally correspond to muscle tissue and E values about 100 kPa generally correspond to collagenous tissue.

Effect of Cell Morphology: Lineage Commitment Influenced by Matrix Stiffness and Dependent on Non-Muscle Myosin II

In the in vitro gel system, on soft, collagen-coated gels that mimic the elasticity of brain tissue (E_{brain}≈0.1-1 kPa), the vast majority of MSCs exhibit a branched morphology (schematically shown in FIG. 1B). This gel system allowed for the control of E through cross-linking, for control of cell adhesion by covalent attachment of collagen-I, and for control of thickness h. The MSC images in FIG. 1B correspond to different substrate elasticities (0.1-1 kPa, 8-17 kPa and 30-40 kPa) at 4 hours and 96 hours. MSCs have a neurite-like branched shape, myoblast-like spindle shape, or osteoblast-like polygonal morphology when cultured on gels in the range of either ˜E_{brain} (0.1-1 kPa), ˜E_{muscle} (8-17 kPa), or the stiffest, collagenous bone-like gels (30-40 kPa), respectively. The branching densities of MSCs approached those of primary neurons on matrigel-coated gels (Flanagan et al., 2000, supra).

In contrast, on 10-fold stiffer substrates that mimic the elasticity of striated muscle (E_{muscle}˜8-17 kPa), MSCs develop myoblast-like, spindle shapes as shown on the graph inset in FIG. 1B. Considerably stiffer substrates (30-40 kPa), that reasonably mimic collagenous bone, yield polygonal MSCs that are similar in shape to osteoblasts. A quantitative analysis of the cell shapes (FIG. 1B, graphs (i) and (ii)) shows that variations in morphology are about the same for MSCs as they are for differentiated cells. Graph (i) shows cell branching per length of primary E 13.5 mouse neurons, MSCs, and blebbistatin-treated MSCs; whereas graph (ii) shows spindle morphology of MSCs, blebbistatin-treated MSCs, and mitomycin C-treated MSCs, as compared to C2C12 myoblasts. Changes in cell shape (<4 days), especially the development of neurite-like branches or spindle-like morphologies, were quantified either by the number of membrane branches per mm of cell, or by a “spindle factor,” the major cell axis/minor cell axis, respectively (see Examples). Furthermore, since the inhibition of proliferation by mitomycin-C (FIG. 1B(i), open squares) has little impact on average cell shape, the morphological results provided herein are consistent with lineage development being a population-level response to substrate elasticity.

For cells on any substrate, blebbistatin was found to block branching, elongation, and any significant spreading of MSCs (FIG. 1B, plots(i) and (ii)). While less specific myosin inhibitors, such as BDM (at mM concentrations), are already known to block neuronal motility (Ruchhoeft et al., J. Neurobiol. 32:567-578 (1997)), as well as the sensitivity of differentiated cells to substrate elasticity (Pelham et al., 1997, supra), blebbistatin appears to be a more selective and potent inhibitor (Straight et al. Science 299:1743-1747 (2003)). For example, blebbistatin inhibits NMM II ATPase activity (at <10 μM drug concentrations) without affecting myosin light chain kinase (“MLCK”), as well as blocking cell blebbing and rapidly disrupting directed cell migration and cytokinesis in vertebrate cells. Myosin crystal structures show that the Mg ATPase binding pocket for blebbistatin relies on the small alanine side chains that are unique to NMM II, isoforms A, B, C, and myosin-VI, which is consistent with the most recent assays of the specificity of blebbistatin (Limouze et al., J. Muscle Res. Cell Motil. 25:337-341 (2004); Straight et al., 2003, supra). Consequently, because MSCs express the three NMM IIs, but no significant myosin-VI, as shown in the present invention, the three NMM IIs and cytoskeleton were strongly implicated in differentiation.

RNA Profiles: Lineage Commitment on Matrices of Tissue-like Stiffness

In an embodiment of the invention, RNA profiles indicated lineage commitment on matrices of tissue-like stiffness. Transcriptional profiles of early neurogenic, myogenic, and osteogenic markers were consistent with lineage identifications based above on morphology. With reference to early passage MSCs, cells on the softest gels (E_{brain}˜0.1-1 kPa) showed the greatest expression of early neurogenic genes (see, for example, FIG. 1C, left column, and Table 3, below). FIG. 1C shows the results of microarray profiling of MSC transcripts in cells cultured on 0.1, 1, 11, and 34 kPa matrices, with the results were normalized to actin levels and then compared to expression of low passage MSCs.

Neuron-specific cytoskeletal markers such as β3-tubulin and neurofilament light chain (“NFL”), as well as adhesion proteins, such as NCAM, all contributed to an average 4-fold up-regulation of the neurogenic transcripts on the softest gels relative to expression on the other gel substrates. In contrast, MSCs grown on E_{muscle}-substrates (11 kPa) expressed 8-fold more myogenic message, with clear up-regulation of relevant transcriptional proteins, such as the Pax activators and myogenic factors (e.g., MyoD). On the stiffest gels (34 kPa), MSCs expressed 3-fold greater osteogenic message, up-regulating osteocalcin and the transcriptional factor CBFα1 (FIG. 1C, middle and right columns, respectively). See also, Engler et al., “Matrix Exasticity is Sensed with Non-Muscle Myosin II and Directs Stem Cell Differentiation,” in press.

Late differentiation genes, such as lineage-specific integrins (α3, α7, β1D) and morphogenetic proteins, are not yet up-regulated (as seen in FIG. 1) relative to initial MSCs, suggesting that in full differentiation, matrix elasticity is likely to couple with other factors, such as soluble factors and other non-collagenous ECM components. Indeed, differentiation marker expression, as elaborated below, appears to average about 50% of control cell levels. This commitment is also lineage specific because transcriptional profiles of early versus late MSCs (up to passage 12) do not differ significantly (see Table 1), even though other investigators have suggested that significant population expansion dramatically alters MSCs.

TABLE 1
oligonucleotide array profiles for genes of mesenchymal origin.

| | | Initial | Expanded | | | | |
|---|---|---|---|---|---|---|---|
| Gene Description | Symbol | MSC | MSC | 0.1 kPa | 1 kPa | 11 kPa | 34 kPa |
| ATP-binding cassette G2 | ABCG2 | 0.60 | 0.51 | 0.31 | 0.42 | 0.58 | 0.14 |
| Alpha-Fetoprotein | AFP | 0.29 | 0.23 | 0.18 | 0.31 | 0.45 | 0.04 |
| [symbol] | [symbol] | [symbol] | [symbol] | [symbol] | [symbol] | [symbol] | [symbol] |
| TAP Binding Protein | TAPBP | 0.25 | 0.30 | 0.39 | 0.42 | 0.52 | 0.22 |
| CD9 Antigen (p24) | CD9 | 0.20 | 0.21 | 0.64 | 0.66 | 0.66 | 0.18 |
| Integrin, Alpha 1 | ITGA1 | 0.70 | 0.51 | 0.20 | 0.19 | 0.16 | 0.34 |
| Integrin, Alpha 2 | ITGA2 | 0.23 | 0.21 | 0.43 | 0.41 | 0.11 | 0.15 |
| Integrin, Alpha 3 | ITGA3 | 0.26 | 0.42 | 0.22 | 0.33 | 0.30 | 0.25 |
| [symbol] | [symbol] | [symbol] | [symbol] | [symbol] | [symbol] | [symbol] | [symbol] |
| Ig Interleukin 1 Receptor | IL-1R | 0.25 | 0.22 | 0.50 | 0.55 | 0.41 | 0.09 |
| [symbol] | [symbol] | [symbol] | [symbol] | [symbol] | [symbol] | [symbol] | [symbol] |
| Interleukin 6 Receptor | IL6R | 0.33 | 0.28 | 0.66 | 0.69 | 0.67 | 0.37 |
| [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |

RNA levels were obtained for initially isolated MSCs (passage 4), as well as MSCs expanded in culture (up to passage 12). MSCs from these groups were plated onto 0.1, 1, 11, and 34kPa matrices, grown for 7 days, and also profiled. Data was normalized to total actin levels and scaled from 0 (no expression) to 1 (maximal expression). The names of genes in bold, italicized type in Table 1 indicate markers that are not generally expressed in the native stem cell population, i.e., normalized expression in initially isolated MSCs<0.15. Notably, there was not a dramatic RNA change between initially isolated and expanded MSCs.

Cytoskeletal Markers and Transcription Factors: Lineage Commitment

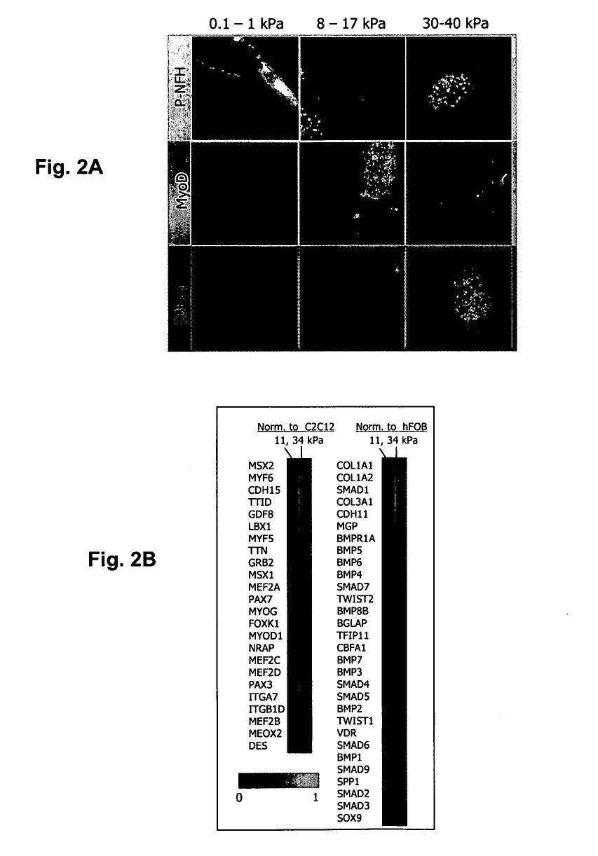

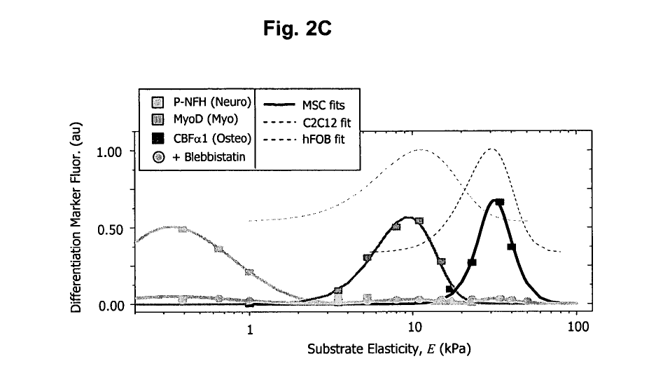

In another embodiment of the invention, cytoskeletal markers and transcription factors can also indicate lineage commitment. FIGS. 2A-2C show protein and transcript profiles as a function of elasticity for mesenchymal stem cells. As shown, protein and transcript profiles are elasticity-dependent under identical media conditions and protein markers are consistent with the E-dependent expression profiling. Blebbistatin blocked all marker expression by MSCs. A majority of cells on the softest, neurogenic matrices expressed the intermediate filament protein phosphorylated neurofilament heavy chain (“P-NFH,” indicated in FIG. 2A by three arrows). This protein is visible in long, branched extensions, but is poorly expressed, if at all, in cells on stiffer gels.

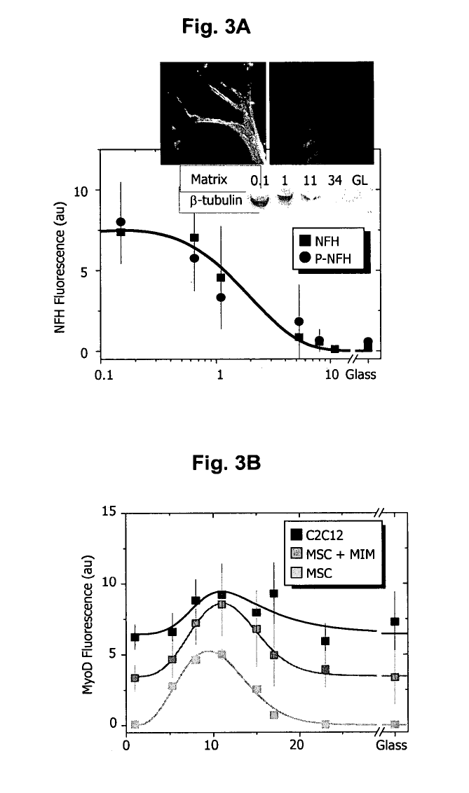

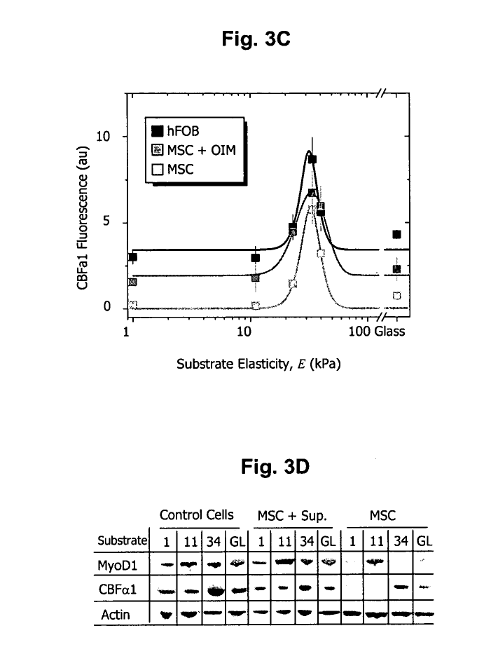

Fluorescence intensity analyses and western immunoblots quantify the elasticity-specific, up-regulation of NFH (neurofilament heavy chain) and P-NFH, as well as β3-tubulin (FIG. 3A). See Examples. In FIGS. 3B and 3C, fluorescent intensities of MyoD1 and CBFα1 in MSCs peaked near E_{muscle} (8-17 kPa) and Eₒₛₜₑₒ (>30 kPa), respectively. However, when differentiation media was substituted with myoblast induction media (“MIM”) or osteoblast induction media (“OIM”), MyoD1 or CBFα1 expression occurred on all substrates, peaking near control cell expression. The depicted solid black line curve in FIG. 3 fits throughout use Equation 3 and also appeared in FIG. 2 with peaks at E*≈0.3 kPa, 10 kPa, 30 kPa and with best fit values for (K, m, and Teff): (2.8·10-4, 2.4, 9.5·10-8), (2.2·10-2, 4.8, 3.4·10-7) (4.2·10-2, 8.1, 1.3·10-6). In FIG. 3D, when normalize to actin, western blots confirmed lineage commitment with matrix or soluble ligand alone, but CBFα1 and MyoD expression only reached control levels when both matrix elasticity and soluble ligand were conducive for differentiation.

Although chemical agonists (Woodbury et al., J. Neurosci. Res. 69:908-917 (2002)) reportedly induce reversible branching in MSCs, ‘branched’ fibroblasts can also be induced chemically (Neuhuber et al., J. Neurosci. Res. 77:192-204 (2004)), which suggests a pan-matrix mechanism with soluble factors. In contrast, primary fibroblasts (FC7) did not branch on the soft elastic substrates (not shown), which implies that matrix stiffness-driven neurogenesis of MSCs is specific to these pluripotent cells, as well as to committed neurons.

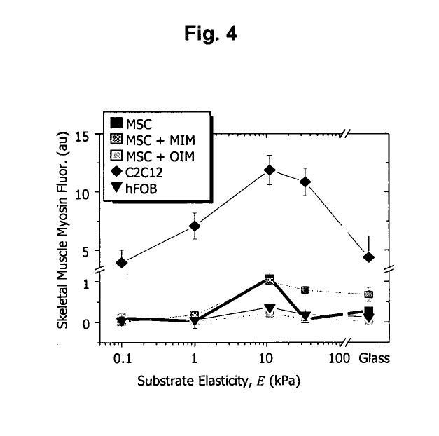

On substrates with stiffness optimal for myogenic differentiation (E_{muscle}≈8-17 kPa), MSCs up-regulate the transcription factor MyoD1, localizing it to the nucleus (large arrow, FIG. 2A). Relative to C2C12 myoblasts, fluorescence intensity analyses indicate 50% expression levels at 1 week. MSCs on softer and stiffer gels do not express MyoD1. Similar normalization of myogenic microarray results of FIG.1 C by the C2C12 myoblast transcripts also shows on average about half the expression and only in cells on E_{muscle}-gels (11 kPa in FIG. 2B; see also Table 3). Myoblast induction media (MIM, Table 2) is known to promote myoblast fusion with expression of skeletal muscle myosin heavy chain (De Bari et al., J. Cell Biol. 160:909-918 (2003)). However, in the present invention, this supplemented media induced expression statistically similar to C2C12 myoblasts, but only for MSCs on E_{muscle}-gels (p=0.45; FIG. 3B). Stimulated by matrix elasticity or soluble factors alone, MyoD western blots (FIG. 3D) and immuno-fluorescence of skeletal muscle myosin confirm myogenic commitment, but incomplete expression of skeletal muscle-specific proteins (FIG. 4).

TABLE 2
Growth conditions for all cell types utilized.

| Media # | Description | Formulation |
|---|---|---|
| MSC GM | MSC Growth Media | Low Glucose DMEM + 20% FBS + |
| | | 1% Penicillin/Streptomycin |
| MSC MIM | Myoblast Induction Media (MIM) | Low Glucose DMEM + 20% FBS + |
| | | 1% Penicillin/Streptomycin + |
| | | 100 nM Dexamethasone + |
| | | 50 μM Hydrocortisone |
| MSC OIM | Osteoblast Induction Media (OIM) | 45% Hank's F12 + 45% α-MEM + 10% FBS + |
| | | 50 μM ascorbate-2-phosphate + 10 mM β- |
| | | glycerol phosphate + |
| | | 100 nM Dexamethasone |
| C2C12 GM | C2C12 Growth Media (GM) | 78% High Glucose DMEM + 20% FBS + |
| FC7 GM | | 1% Chicken Embryo Extract + |
| | | 1% Penicillin/Streptomycin |
| hFOB GM | hFOB Growth Media (GM) | 45% Hank's F12 + 45% DMEM + 10% FBS |

All MSC cultures use MSC GM for all experiments, except when noted.

Table 2 shows oligonucleotide array profiles for MSCs cultured on 0.1, 1, 11, and 34 kPa matrices that were normalized to actin levels, and were differentially compared to gene up-regulation (+1) to maximum down-regulation (−1). mRNA expression for MSCs cultured on 11 and 34 kPa matrices were also expressed as a fraction of mRNA expression in C2C 12 myoblasts or hFOB osteoblasts for the genes as indicated (scale: 0 to 1).

TABLE 3
Growth conditions for all cell types utilized.

| | | | | (0.1 kPa | (1 kPa | (11 kPa | | | |
|---|---|---|---|---|---|---|---|---|---|
| | | | | MSC) − | MSC) − | MSC) − | (34 kPa | (11 kPa | (34 kPa |
| Gene Description | | GenBank/ | Lineage | MSC | MSC | MSC | MSC) − MSC | MSC) | MSC) |
| (Normalization) | Symbol | Unigene # | Marker | MSC | MSC | MSC | MSC | C2C12 | hFOB |
| Microtubule-Assoc. | MAPT | Hs.101174 | Neuro | 0.51 | 0.59 | 0.35 | 0.05 | | |
| Protein Tau | | | | | | | | | |
| Tau Tubulin Kinase 1 | TTBK1 | NM_032538.1 | Neuro | 0.48 | 0.56 | 0.32 | 0.08 | | |
| Tau Tubulin Kinase 2 | TTBK2 | NM_173500.2 | Neuro | 0.29 | 0.35 | 0.24 | 0.07 | | |
| Tubulin, Alpha 3 | TUBA3 | NM_006009 | Neuro | 0.11 | 0.02 | −0.08 | −0.48 | | |
| Tubulin, Beta 1 | TUBB1 | NM_030773 | Neuro | 0.83 | 0.70 | 0.39 | 0.05 | | |
| Tubulin, Beta 3 | TUBB3 | NM_006086 | Neuro | 0.53 | 0.24 | 0.05 | 0.02 | | |
| Tubulin, Beta 4 | TUBB4 | NM_006087 | Neuro | 0.85 | 0.77 | 0.28 | −0.28 | | |
| Glial Der. Neurotrophic | GDNF | NM_000514 | Neuro | 1.00 | 0.77 | 0.16 | 0.01 | | |
| Factor | | | | | | | | | |
| GDNF Receptor Alpha 1 | GFRA1 | NM_005264 | Neuro | 0.47 | 0.38 | 0.09 | 0.01 | | |
| N-Cadherin | CDH2 | NM_001792 | Neuro | 0.13 | 0.33 | 0.51 | 0.06 | | |
| TNF Receptor Member 5 | CD40 | NM_001250 | Neuro | 0.31 | 0.49 | 0.26 | 0.10 | | |
| TNF Receptor, Member 6 | FAS | NM_152877 | Neuro | 0.38 | 0.50 | 0.30 | 0.10 | | |
| Brain-Der. Neurotrophic | BDNF | NM_001709 | Neuro | 0.37 | 0.42 | 0.01 | 0.03 | | |
| Factor | | | | | | | | | |
| Neurofilament Light | NEFL | NM_006158 | Neuro | 0.30 | 0.28 | 0.09 | 0.02 | | |
| Chain | | | | | | | | | |
| Internexin Neuronal IF | INA | NM_032727.2 | Neuro | 0.62 | 0.46 | 0.14 | 0.03 | | |
| Alpha | | | | | | | | | |
| Nerve Growth Factor | NGF | NM_002506 | Neuro | 0.39 | 0.31 | 0.13 | 0.07 | | |
| Beta | | | | | | | | | |
| Neuregulin 1 | NRG1 | NM_013957 | Neuro | 0.38 | 0.33 | 0.19 | 0.05 | | |
| Signal Activator of | STAT3 | NM_003150 | Neuro | 0.40 | 0.39 | 0.10 | 0.06 | | |
| Transcript. 3 | | | | | | | | | |
| Nestin | NES | NM_006617.1 | Neuro | 0.26 | 0.29 | 0.04 | 0.11 | | |
| Neural CAM 1 | NCAM1 | NM_000615 | Neuro | 0.74 | 0.32 | 0.15 | 0.13 | | |
| Integrin, Beta 3 | ITGB3 | NM_000212.2 | Neuro | −0.57 | −0.41 | −0.58 | −0.60 | | |
| Paired Box Gene 3 | PAX3 | NM_181457.1 | Myo | 0.34 | 0.30 | 0.30 | 0.11 | 0.56 | |
| Paired Box Gene 7 | PAX7 | Hs.113253 | Myo | 0.16 | 0.14 | 0.56 | 0.05 | 0.59 | |
| Myogenic Factor 3 | MYOD1 | NM_002478.3 | Myo | 0.29 | 0.18 | 0.79 | 0.10 | 0.58 | |
| Myogenic Factor 4 | MYOG | Hs.2830 | Myo | 0.12 | 0.11 | 0.48 | 0.06 | 0.59 | |
| Myogenic Factor 5 | MYF5 | NM_005593 | Myo | 0.17 | 0.22 | 1.00 | 0.09 | 0.65 | |
| Myogenic Factor 6 | MYF6 | NM_002469.1 | Myo | 0.12 | 0.16 | 0.49 | 0.08 | 0.76 | |
| Mesenchyme Homeobox 2 | MEOX2 | Hs.527007 | Myo | 0.14 | 0.22 | 0.57 | 0.12 | 0.46 | |
| Forkhead Box K1 | FOXK1 | Hs.520634 | Myo | 0.51 | 0.45 | 0.73 | 0.13 | 0.59 | |
| Myostatin | GDF8 | NM_005259 | Myo | 0.16 | 0.14 | 0.39 | 0.10 | 0.67 | |
| MADS Enhancer Factor | MEF2A | NM_005587 | Myo | 0.33 | 0.20 | 0.48 | 0.09 | 0.62 | |
| 2A | | | | | | | | | |
| MADS Enhancer Factor | MEF2B | NM_005919 | Myo | 0.60 | 0.24 | 0.49 | 0.12 | 0.53 | |
| 2B | | | | | | | | | |
| MADS Enhancer Factor | MEF2C | NM_002397 | Myo | 0.21 | 0.07 | 0.58 | 0.08 | 0.57 | |
| 2C | | | | | | | | | |
| MADS Enhancer Factor | MEF2D | NM_005920 | Myo | 0.16 | 0.06 | 0.77 | 0.13 | 0.57 | |
| 2D | | | | | | | | | |
| Growth Factor Bound | GRB2 | NM_002086 | Myo | 0.03 | 0.00 | 0.31 | 0.06 | 0.64 | |
| Receptor 2 | | | | | | | | | |
| Desmin | DES | NM_001927.3 | Myo | 0.14 | 0.13 | 0.50 | 0.15 | 0.46 | |
| Msh Homeobox | MSX1 | NM_002448.1 | Myo | 0.13 | 0.12 | 0.39 | 0.09 | 0.63 | |
| Homolog 1 | | | | | | | | | |
| Msh Homeobox | MSX2 | NM_002449 | Myo | 0.05 | 0.05 | 0.31 | 0.06 | 1.00 | |
| Homolog 2 | | | | | | | | | |
| Ladybird Homeobox | LBX1 | NM_006562.3 | Myo | −0.42 | −0.43 | 0.05 | 0.01 | 0.66 | |
| Homolog 1 | | | | | | | | | |
| Nebulin-Rel. Anchoring | NRAP | Hs.268788 | Myo | 0.11 | 0.09 | 0.38 | 0.09 | 0.58 | |
| Protein | | | | | | | | | |
| Myotilin | TTID | Hs.84665 | Myo | 0.17 | 0.08 | 0.67 | 0.23 | 0.67 | |
| Titin | TTN | NM_003319.2 | Myo | 0.12 | 0.01 | 0.38 | 0.05 | 0.65 | |
| M-Cadherin | CDH15 | NM_004933 | Myo | 0.15 | 0.02 | 0.60 | 0.28 | 0.75 | |
| Integrin, Beta 1D | ITGB1D | NM_033668.1 | Myo | 0.20 | 0.12 | 0.70 | 0.16 | 0.54 | |
| Integrin, Alpha 7 | ITGA7 | NM_002206 | Myo | −0.89 | −0.87 | 0.52 | −0.20 | 0.55 | |
| Core Binding Factor | CBFA1 | NM_004348 | Osteo | −1.00 | −0.87 | 0.06 | 0.00 | | 0.37 |
| Alpha 1 | | | | | | | | | |
| Cadherin 11, Type 2 | CDH11 | NM_001797 | Osteo | −0.09 | 0.03 | 0.11 | 0.17 | | 0.69 |
| Osteopontin | SPP1 | NM_000582 | Osteo | −0.61 | −0.67 | −0.14 | 0.05 | | 0.36 |
| Tuftelin Interacting | TFIP11 | NM_012143 | Osteo | 0.03 | −0.24 | 0.09 | 0.15 | | 0.48 |
| Protein 11 | | | | | | | | | |
| Twist Homolog 1 | TWIST1 | NM_000474 | Osteo | 0.03 | −0.31 | 0.18 | 0.15 | | 0.43 |
| Twist Homolog 2 | TWIST2 | NM_057179 | Osteo | 0.05 | 0.01 | 0.11 | 0.25 | | 0.49 |
| Sex Det. Region Y-box 9 | SOX9 | NM_000346 | Osteo | 0.11 | 0.09 | 0.37 | 0.41 | | 0.32 |
| SMAD, Mothers Against | SMAD1 | NM_005900 | Osteo | 0.10 | 0.15 | 0.16 | 0.15 | | 0.81 |
| DPP 1 | | | | | | | | | |
| SMAD, Mothers Against | SMAD2 | NM_005901 | Osteo | 0.08 | 0.12 | 0.21 | 0.57 | | 0.36 |
| DPP 2 | | | | | | | | | |
| SMAD, Mothers Against | SMAD3 | NM_005902 | Osteo | 0.13 | 0.12 | 0.20 | 0.20 | | 0.34 |
| DPP 3 | | | | | | | | | |
| SMAD, Mothers Against | SMAD4 | NM_005359 | Osteo | 0.13 | 0.15 | 0.18 | 0.17 | | 0.44 |
| DPP 4 | | | | | | | | | |
| SMAD, Mothers Against | SMAD5 | NM_005903 | Osteo | 0.12 | 0.08 | 0.28 | 0.15 | | 0.43 |
| DPP 5 | | | | | | | | | |
| SMAD, Mothers Against | SMAD6 | NM_005585 | Osteo | 0.17 | 0.13 | 0.29 | 0.44 | | 0.42 |
| DPP 6 | | | | | | | | | |
| SMAD, Mothers Against | SMAD7 | NM_005904 | Osteo | 0.31 | 0.39 | 0.37 | 0.53 | | 0.50 |
| DPP 7 | | | | | | | | | |
| SMAD, Mothers Against | SMAD9 | NM_005905 | Osteo | 0.17 | 0.26 | 0.14 | 0.46 | | 0.36 |
| DPP 9 | | | | | | | | | |
| Vitamin D receptor | VDR | NM_000376 | Osteo | 0.18 | 0.22 | 0.14 | 0.17 | | 0.42 |
| Osteocalcin | BGLAP | NM_199173.2 | Osteo | 0.19 | 0.21 | 0.22 | 0.20 | | 0.48 |
| Bone Morphogenetic | BMP1 | NM_006129 | Osteo | 0.27 | 0.37 | 0.38 | 1.00 | | 0.41 |
| Protein 1 | | | | | | | | | |
| Bone Morphogenetic | BMP2 | NM_001200 | Osteo | 0.32 | 0.40 | 0.30 | 0.22 | | 0.43 |
| Protein 2 | | | | | | | | | |
| Bone Morphogenetic | BMP3 | NM_001201 | Osteo | 0.14 | 0.20 | 0.14 | 0.16 | | 0.44 |
| Protein 3 | | | | | | | | | |
| Bone Morphogenetic | BMP4 | NM_130851 | Osteo | 0.26 | 0.36 | 0.19 | 0.28 | | 0.54 |
| Protein 4 | | | | | | | | | |
| Bone Morphogenetic | BMP5 | NM_021073 | Osteo | 0.21 | 0.41 | 0.26 | 0.22 | | 0.63 |
| Protein 5 | | | | | | | | | |
| Bone Morphogenetic | BMP6 | NM_001718 | Osteo | 0.19 | 0.39 | 0.17 | 0.25 | | 0.54 |
| Protein 6 | | | | | | | | | |
| Bone Morphogenetic | BMP7 | NM_001719 | Osteo | 0.09 | 0.23 | 0.02 | 0.13 | | 0.45 |
| Protein 7 | | | | | | | | | |
| Bone Morphogenetic | BMP8B | NM_001720 | Osteo | 0.10 | 0.17 | 0.10 | 0.10 | | 0.49 |
| Protein 8B | | | | | | | | | |
| Bone Morpho. Protein | BMPR1A | NM_004329 | Osteo | 0.07 | 0.20 | 0.10 | 0.10 | | 0.64 |
| Recptr 1A | | | | | | | | | |
| Matrix Gla Protein | MGP | NM_000900 | Osteo | 0.08 | 0.17 | 0.06 | 0.06 | | 0.68 |
| Collagen, Type 1, Alpha 1 | COL1A1 | NM_000088 | Osteo | 0.10 | 0.14 | 0.06 | 0.04 | | 1.00 |
| Collagen, Type 1, Alpha 2 | COL1A2 | NM_000089 | Osteo | −0.30 | −0.05 | −0.67 | 0.01 | | 1.00 |
| Collagen, Type 3, Alpha 1 | COL3A1 | NM_000090 | Osteo | −0.44 | −0.16 | −0.80 | 0.00 | | 0.71 |
| Myosin IA | MYO1A | NM_005379.2 | Myosin | 0.30 | 0.35 | 0.12 | 0.12 | 0.61 | 0.74 |
| Myosin IIIB | MYO3B | NM_138995.1 | Myosin | −0.67 | −0.45 | 0.04 | 0.02 | 0.73 | 0.45 |
| Myosin VB | MYO5B | Hs.550481 | Myosin | 0.09 | 0.07 | 0.27 | 0.13 | 0.65 | 0.94 |
| Myosin IXa | MYO9A | NM_006901.1 | Myosin | −0.82 | −0.72 | 0.04 | 0.03 | 0.67 | 0.65 |
| Myosin VI | MYO6 | Hs.302051 | Myosin | 0.06 | 0.06 | 0.15 | 0.06 | 0.66 | 0.56 |
| Myosin Light Chain III | MYL6 | Hs.505705 | Myosin | 0.06 | 0.06 | 0.12 | 0.08 | 0.67 | 0.48 |
| Myosin Heavy Chain IIa | MHC2a | NM_017534.3 | Myosin | −0.39 | −0.48 | −0.02 | 0.00 | 0.64 | 0.56 |
| Sk. Muscle Myosin | MYH3 | Hs.440895 | Myosin | 0.24 | 0.17 | 0.29 | 0.18 | 0.62 | 0.65 |
| Heavy III | | | | | | | | | |
| Non-Muscle Myosin IIA | NMM2A | Hs.16355 | Myosin | −0.52 | −0.44 | 0.01 | 0.00 | 0.83 | 0.55 |
| Non-Muscle Myosin IIB | NMM2B | Hs.467142 | Myosin | 0.13 | 0.11 | 0.23 | 0.09 | 0.69 | 0.34 |
| Non-Muscle Myosin IIC | NMM2C | NM_002473 | Myosin | 0.31 | 0.26 | 0.35 | 0.17 | 0.57 | 0.57 |
| Human Myosin Light | HML2B | NM_013292 | Myosin | 0.34 | 0.25 | 0.53 | 0.13 | 0.58 | 0.66 |
| Chain IIB | | | | | | | | | |
| Talin 1 | TLN1 | Hs.375001 | Adhesion | 0.02 | 0.03 | 0.14 | 0.07 | 0.72 | 0.60 |
| Filamin C, Gamma | FLNC | Hs.58414 | Adhesion | −0.25 | −0.21 | 0.00 | 0.02 | 0.70 | 0.54 |
| Paxillin | PXN | NM_002859 | Adhesion | −0.23 | −0.26 | 0.00 | 0.02 | 0.62 | 0.54 |
| Vinculin | VCL | NM_003373 | Adhesion | 0.04 | 0.02 | 0.04 | 0.06 | 0.62 | 0.51 |
| Laminin, Alpha 2 | LAMA2 | NM_000426 | Adhesion | 0.37 | 0.40 | 0.39 | 0.31 | 0.77 | 0.77 |
| Actinin, Alpha 1 | ACTN1 | NM_001102 | Adhesion | −0.03 | 0.01 | 0.04 | 0.01 | 0.60 | 0.55 |
| PTK2 Protein Tyrosine | PTK2 | NM_005607.3 | Signaling | −0.59 | −0.50 | 0.02 | 0.01 | 0.72 | 0.59 |
| Kinase | | | | | | | | | |
| Ras Homolog A | RHOA | Hs.247077 | Signaling | −0.26 | −0.26 | 0.02 | 0.02 | 0.59 | 0.53 |
| Ras-C3 Botulinum | RAC1 | NM_006908.3 | Signaling | −0.37 | −0.20 | 0.02 | 0.01 | 0.69 | 0.45 |
| Substrate 1 | | | | | | | | | |
| Cell Division Cycle 42 | CDC42 | NM_001791 | Signaling | 0.18 | 0.10 | 0.02 | 0.17 | 0.78 | 0.52 |
| Rho-Assoc. Coil-Coil | ROCK1 | NM_005406 | Signaling | 0.05 | 0.06 | 0.02 | 0.05 | 0.95 | 0.53 |
| Kinase 1 | | | | | | | | | |
| Diaphanous Homolog 2 | DIAPH2 | NM_006729.2 | Signaling | 0.02 | −0.28 | 0.00 | 0.10 | 0.63 | 0.45 |
| Striated Muscle | STARS | Hs.374668 | Signaling | 0.22 | 0.20 | 0.34 | 0.07 | 0.96 | 0.39 |
| Activator-Rho | | | | | | | | | |

All MSC cultures use MSC GM for all experiments, except when noted.

In Table 3, oligonucleotide array profiles for MSCs cultured on 0.1, 1, 11, and 34 kPa matrices were normalized to actin levels, differentially compared to gene expression of low (4) passage MSCs, and scaled from maximum up-regulation (+1) to maximum down-regulation (−1). mRNA expression for MSCs cultured on 11 and 34 kPa matrices were also expressed as a fraction of mRNA expression in C2C12 myoblasts or hFOB osteoblasts for those indicated genes (scale: 0 to 1).

For osteogenesis, expression of the transcription factor CBFα1 is a first step since its inhibition limits osteogenesis (Gilbert et al., 2002, supra); Salim et al., 2004, supra). On osteogenic gel substrates (Eₒₛₜₑₒ≈30-40 kPa) and with standard growth media, MSCs expressed CBFα1 (see FIG. 2A, open arrow) while MSCs on the softer gels did not. Fluorescence intensity analyses again showed half the expression level of the osteoblasts, consistent with osteogenic microarray averages after normalization by hFOB-osteoblast expression (34 kPa in FIG. 2B; see also Table 3, above). When cultured in osteoblast induction media (OIM), increased CBFα1 expression was observed in MSCs grown on all substrates, and showed, as do osteoblasts, optimum differentiation on these stiff, osteogenic gels (p=0.4) (see FIGS. 3C and 3D). Elasticity-directed marker expression on the various substrates is summarized in FIG. 2C, wherein each lineage commitment optimum is also fitted to a three-parameter, chemo-mechanical model predicated on substrate elasticity and contractility as described below.

MSCs Couple NMM II Expression to Matrix Stiffness

In yet another embodiment of the invention, MSCs couple NMM II expression to matrix stiffness. Although, cellular contractility and related signals have been postulated by others (Bick et al., Cell Adhes. Commun. 6:301-310 (1998); Engler et al., 2004c, supra; McBeath et al., 2004, supra; Muller et al., Biochem. Biophys. Res. Commun. 229:198-204 (1996); Puceat et al., Mol. Biol. Cell 14:2781-2792 (2003)) to influence differentiation, the prior art includes no report or evidence of any kind that has suggested the effect of strong, tissue-directed feedback of matrix elasticity on lineage commitment of previously pluripotent stem cells. Chronic treatment of MSCs on various gels with blebbistatin blocks expression of neurogenic, myogenic, and osteogenic markers (FIG. 2C). See also Engler et al., supra in press.

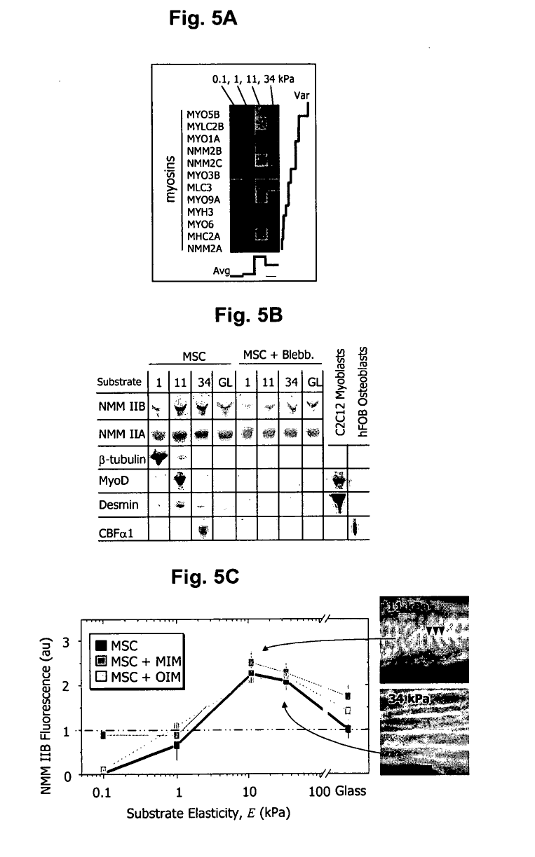

Consistent with myosin regulation by substrate elasticity, a number of myosin RNAs are up-regulated on stiffer gels (11, 34 kPa) when compared to the softer matrices (FIG. 5A). Western blots and immunofluorescence show NMM IIB is up about two-fold relative to myosin levels before differentiation (FIGS. 5B and 5C), whereas expression is suppressed on the softest substrates, 0.1-1 kPa. Both myosins and the various markers for differentiation showed stiffness sensitivity and inhibition by blebbistatin. The induction media (MIM or OIM) had comparatively little effect on these E-responsive expression profiles (FIG. 5C).

Select myosin genes appear more matrix sensitive than others, based on clustering of microarray data by variation with E (FIG. 5A). Western blots of non-muscle myosins confirm the variation, that is, NMM IIB expression is more sensitive to matrix elasticity than NMM IIA expression (FIG. 5B). The blots also confirm (i) myogenic commitment with requisite up-regulation of both MyoD and the intermediate filament protein, desmin (Weitzer et al., Dev. Biol. 172:422-439 (1995)) and (ii) osteogenic commitment with CBFα1 . Intensity analyses of immunofluorescence images of NMM IIA (FIG. 5C) reinforce microarray and blot results and further reveal changes in myosin organization.

On soft, E_{brain}-gels, non-muscle myosin staining was diffuse; while on stiff E_{muscle}-gels, myosin striations emerged. Spacing between these nascent striations is the same for MSCs and age-matched C2C12 myocytes (1.0±0.3 μm) and is consistent with non-muscle myosin organization (Verhovsky et al., J. Cell Biol. 131:989-1002 (1995)), as well as periodic β3-integrin clusters (Giannone et al., Cell 116:431-443 (2004)) in embryonic fibroblasts. However, the striation period was smaller than the spacing set by myogenic molecular “rulers,” such as titin (TTN) (see FIG. 2B) (Golson et al., Cell Motil. Cytoskeleton 59:1-16 (2004); Sanger et al., Clin. Ortho. Rel. Res. 403S:S153-S162 (2002)), indicating that MSCs, and even C2C12 cells, have not fully assembled mature myofibrils, even after a week in culture. These striations were lost, however, on the stiffest Eₒₛₜₑₒ-gels, where stress fibers predominate.

Chronic blebbistatin inhibition of non-muscle myosin II activity also decreased NMM IIB expression about 10-fold, to levels comparable to MSCs on soft gels (see FIG. 5B). NMM IIA expression was only slightly reduced with blebbistatin, but both striation and stress fiber formation were suppressed, consistent with a relaxation effect (Griffin et al., J Cell Sci. 117:5855-5863 (2004)). On soft gels, cells also generally lack cytoskeletal organization, as reinforced with results below. NMM IIB controls fibroblast guidance (Lo et al., 2004, supra), cardiomyocyte function (Takeda et al., 2003, supra), and neurite out-growth (Bridgman et al., J. Neurosci. 21:6159-6169 (2001); Ma et al., Mol. Biol. Cell 15:2568-2579 (2004); Rochlin et al., J. Cell Sci. 108(Pt 12):3661-3670 (1995)). As a result, it is not surprising that blebbistatin treatment not only inhibits MSC morphogenesis (FIG. 1B, graph (i) and (ii)), but that it also inhibits expression of the lineage markers (FIG. 5B), which is consistent with non-muscle myosin II activity ultimately regulating differentiation profiles in addition to its own expression. See also Engler et al., supra, in press.

Rho GTP-ases Switch Signals for Lineage Specification

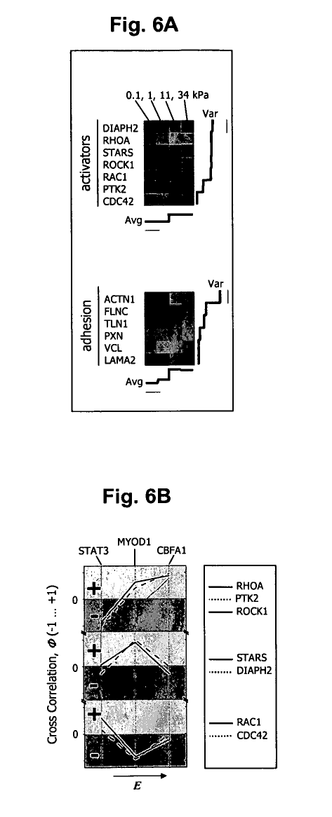

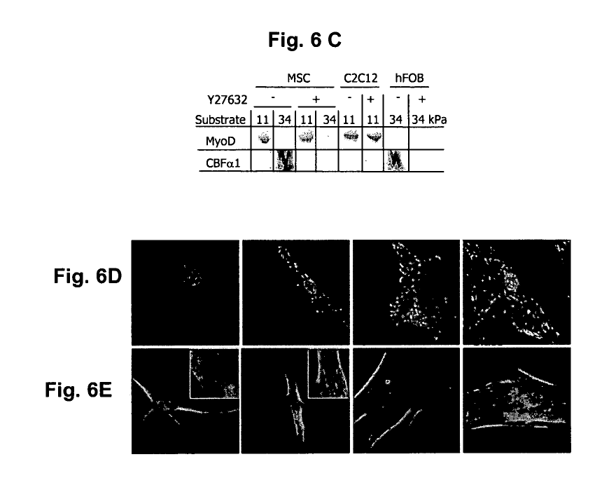

In an embodiment of the invention, Rho GTP-ases switched signals for lineage specification. For example, representative activator/effector and cell adhesion transcripts reveal a range of myosin-based sensitivities to substrate elasticity. Although myosin showed on average a peak expression in stem cells on E_{muscle}-gels (FIGS. 5A-5C), the average trend with the representative activator and adhesion transcripts is a monotonic increase versus substrate-E (“Avg” in graphs of FIG. 6A). RhoA shows the strongest increase with E, followed by PTK2 (or focal adhesion kinase (FAK)), but overall expression is highly variable. Rho and PTK2 (FAK) correlated positively with both myogenesis and osteogenesis, while the Rho-GTPase, ROCK, correlated only with osteogenesis. Rho GTP-ases DIAPH2 (mDia homolog) and STARS correlate only with myogenesis, whereas Ras superfamily proteins, Rac1 and Cdc42, correlated positively only with neurogenesis.

Variance (Var) versus substrate-E spans an order of magnitude with RhoA and the mDiaphanous (mDia) homolog, DIAPH2 (Watanabe et al., Embo J. 16:3044-3056 (1992)), showing the largest variance. Since Ras and Rho family activators and their effectors generally regulate cytoskeleton, contractility, adhesion, and transcription (Kaibuchi et al., Annu. Rev. Biochem. 68:459-486 (1999)), relatively low RhoA expression on soft, neurogenic substrates appears consistent with low myosin activity (FIGS. 5A-5C). Additionally, in the developing brain, RhoA is spatially regulated and expressed only where cell division/proliferation occurs (Olenik et al., Brain Res. Mol. Brain Res. 70:9-17 (1999)). Peak expression of both DIAPH2 and STARS (Striated Muscle Activator-Rho) on E_{muscle}-gels (11 kPa) is consistent with these two proteins having a prominent role in myogenesis (Arai et al., J. Biol. Chem. 277:24453-24459 (2002); Gopinath et al., in press; Kuwahara et al., Mol. Cell Biol. 25:3173-3181 (2005)).

To better identify lineage-specific activators of key transcription factors, a crosscorrelation function, Φ, was developed to compare the elasticity-dependent expression of each activator/effector gene with key transcription factor genes. These are respectively denoted as gene x(E) and gene y(E) in the function:

$$Φ = \frac{\sum ( x - \overline{x} ) ( y - \overline{y} )}{\sqrt{\sum {( x - \overline{x} )}^{2} {( y - \overline{y} )}^{2}}} \qquad Equation 1$$

A value of Φ=1 indicates that genes x and y have identical elasticity-dependence, i.e., expression between genes is similar regardless of microenvironment, whereas a value of Φ=−1 indicates an inverse correlation, i.e., gene expression trends are highly variable across different matrices.

Pathway divergence is apparent in FIG. 6B from the cross-correlations of the stiffness-dependent expression of activators/effectors with lineage-specific transcription factors STAT3 (neurogenic lineage; Fukuda et al., Anat. Sci. Int. 80:12-18 (2005)), MYOD1 (myogenic), and CBFα1 (osteogenic). While RhoA, PTK2 (i.e., FAK), and ROCK1 all show inverse correlations with neurogenesis, they all correlate positively with osteogenesis (i.e., CBFα1 ). These three regulators thus have the strongest positive influence on stiff materials where the cells are strongly anchored and exert strong contractions on their substrates (see below), consistent with osteogenic development of MSCs. It should be noted that RhoA and PTK2 are also positively correlated with myogenic commitment, while ROCK is not, indicating the importance of Rho GTPases.

Cell adhesions often rely on the RhoGTP-ases, effectors ROCK and mDia, to promote cytoskeletal assembly. ROCK is typically present in large, tension-generating processes associated with long, narrow focal adhesions (Riveline et al., 2001, supra) and cell shape maintenance, as well as osteogenesis (McBeath et al., 2004, supra). Myoblasts, on the other hand, can differentiate when ROCK is inhibited, but require RhoA activity via serum response factor (“SRF”) (Dhawan et al., J. Cell Sci. 117:3735-3748 (2004)), and other effectors. STARS (striated muscle activator of Rho signaling), and the formin homology protein, mDia, respectively bind actin and promote its assembly, activating SRF, but dependent on Rho (Copeland et al., Mol. Biol. Cell 13:4088-4099 (2002); Kuwahara et al., 2005, supra).

The sensitivity of adhesions to force depends on mDia (not ROCK), which seems likely to foster both the labile and punctate focal contacts of myogenesis seen in FIG. 6D, as well as a structurally distinct cytoskeleton poised for striation (FIG. 5C) and myofibrillogenesis. Addition of the ROCK inhibitor Y27632 to cell cultures for 1 week prior to western blotting (FIG. 6C) showed that MyoD expression by MSCs and C2C12 myoblasts on permissive matrices was not affected. This is consistent with the expected mDia-ROCK pathway switch in lineage commitment of MSCs. However, with MSCs and hFOB-osteoblasts on substrates with osteogenic stiffness, inhibiting ROCK blocked expression of CBFα1. ROCK inhibition is thus similar to blebbistatin treatment, but more lineage-selective.

While Rac1 is described as a pleiotropic regulator of cell adhesion and the cytoskeleton (Benitah et al., 2005, supra), both Rac1 and Cdc42 showed high activity in actin-driven motile processes, as compared to myosin-dominated contractility (Bershadsky et al., 2003, supra). The former processes predominate in growth cone migration (Sakumura et al., Biophys. J. 89:812-822 (2005)). In the present invention, RAC1 and, to a lesser extent, CDC42 showed a positive correlation with neurogenesis (FIG. 6B), consistent with the low myosin expression on soft gels (FIGS. 4). Nonetheless, since blebbistatin blocks all differentiation, including neurogenesis, myosin-based contractile mechanisms are broadly important in matrix-directed differentiation.

Stem Cell Differentiation: Adhesion and Contractility Balance, but Increase with Matrix Stiffness

Like the matrix-directed variation of activators, select focal adhesion transcripts, such as non-muscle α-actinin, filamin, and talin also appear to be particularly stiffness-sensitive and driven in expression by the stiff substrates (FIG. 6A(bottom)). In contrast, collagen and laminin transcripts appear relatively uninfluenced in these elastic matrix culture systems (FIGS. 1C and 6A(top)). Without wishing to be so bound by any hypothesis, this seems likely due to the fact that collagen is already attached to a matrix of specified stiffness. As a result, MSCs simply respond to the matrix, rather than remodel it. Previous work with collagen-I coated gels, indeed, shows that above a threshold level of adhesive ligand, spreading of tissue cells and their cytoskeletal organization is relatively insensitive to ligand (Engler et al., Surface Science 570:142-154 (2004b)).

Consistent with the transcription profiles for activators/effectors and adhesions, as well as the earliest reports of substrate-stiffness responses (Engler et al., 2004a, supra; Gaudet et al., Biophys. J. 85:3329-3335 (2003), stiff substrates were found to promote paxillin integration in the growth and elongation of focal adhesions (FIG. 6D). Rigidification of cell-derived three-dimensional (3D) matrices altered 3D-matrix adhesions, and the adhesions were replaced by large, nonfibrillar focal adhesions similar to those found on fixed 2D substrates of fibronectin (Cukierman et al., 2001, supra). Consistent with a role for signaling in stiffness sensing, tyrosine phosphorylation on multiple proteins (including paxillin) appears to be broadly enhanced in cells on stiffer gel substrates; whereas, pharmacologically induced, nonspecific hyperphosphorylation drives focal adhesion formation on soft materials.

Actomyosin is the contractile element of the myotubule. On very soft gels that are micropattemed with collagen strips so as to generate well-separated myotubes, actomyosin appeared diffuse after weeks in culture. However, on very stiff gels, as well as on glass micropatterns, stress fibers and strong focal adhesions predominate, suggesting a state of isometric contraction. Notably, on gels with an elasticity that approximates that of relaxed muscle bundles (E˜10 kPa), a large fraction of myotubes in culture exhibited definitive actomyosin striations. Actomyosin striation is even more prominent when cells are cultured on top of a first layer of muscle cells (as shown in Discher et al., 2005, supra). The lower myotubes attached strongly to glass and formed abundant stress fibers: whereas the upper myotubes differentiated to the more physiological, striated state. Although cell-cell contact may provide additional signals, the elasticity E of the myotubes, as measured by atomic force microscopy, was in the same range as that of gels that are optimal for differentiation, and importantly, was in the same range as that of normal muscle tissue.

Cell-cell contact appears to induce similar cell-on-gel effects for systems other than muscle. Astrocytes growing on glass, for example, appeared to provide a soft cell “stroma” adequate for neuronal branching that is similar to gels having brainlike E. Cell-cell contact may have a similar effect when cells are grown at a high density. When endothelial cells are confluent, the cells have indistinguishable morphologies on soft versus stiff substrates, whereas cells attached only to an underlying stiff surface differ in their spreading and cytoskeletal organization. Related results are also emerging from the present invention using epithelial cells and fibroblasts, as well as cardiomyocytes, showing a tendency to aggregate and form cell-cell contacts in preference to contact with soft gels.

Inhibition of actomyosin contractions largely eliminated prominent focal adhesions, whereas stimulation of contractility drives integrin aggregation into adhesions (Chrzanowska-Wodnicka et al., 1996, supra). Additionally, although microtubules have been proposed to act as “struts” in cells, and thus limit wrinkling of thin films by cells (Pletjushkina et al., Cell Motil. Cytoskeleton 48:235 (2001)), quantification of their contributions to cells on gels shows that they provide only a minor fraction of the resistance (14%) to contractile tensions. Most of a cell's tension is thus resisted by matrix (Wang et al., Proc. Natl. Acad. Sci. USA 98:7765 (2001)). On the stiffest, osteogenic gels (34 kPa), represented by thin gels of h≈0.5 μm, the adhesions were long and thin and slightly more peripheral than they appear on glass (FIG. 6D). Actin assembly followed (FIG. 6E), which generalizes the E-driven assembly of the cytoskeleton to MSCs. Consistent with this, NMM-II is already known to influence focal adhesions (Conti et al., J. Biol. Chem. 279:41263-41266 (2004)).

Materials ranging from fibrin gels and microfabricated pillars to layer-by-layer polymer assemblies (Georges et al., Appl. Physiol. 98:1547 (2005); Raeber et al., Biophys. J. 89:1374 (2005); Saez et al., in press; Wang et al, in press; Engler et al., 2004b, supra)), all suggest a similar trend of more organized cytoskeleton and larger, more stable adhesions with increasing E as outlined in the present invention, despite likely differences in adhesive ligand density and long-time elasticity. However, the responses appear to be specific to anchorage-dependent and/or relatively contractile cells.

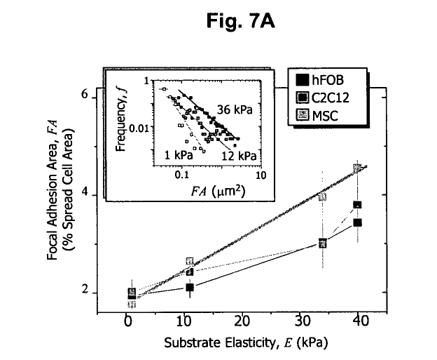

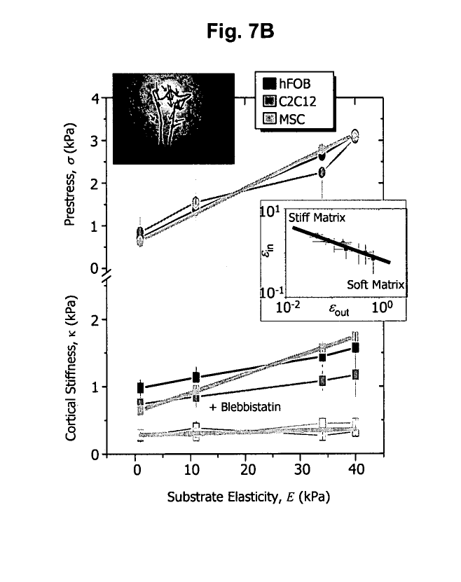

To assess adhesions and to begin addressing length scales of “micro” environments, e.g., the volume of the environment that interacts with the cell, which is on the scale of nanometers to microns, thin polyacrylamide (PA) gels were cast with spacer beads to a thickness of h˜500 nm. This length scale allowed for low-intensity total internal reflectance fluorescence (TIRF) microscopy (Axelrod et al., J. Microsc. 129(Pt 1):19-28 (1983)). MSCs plated on thin, but stiff, matrices spread more with many more large focal adhesions (see FIGS. 6D and 7A). Yet from the viewpoint of a cell, a thin soft gel on glass is perceived as having an apparent elastic modulus greater than the gel modulus due to the proximity of the rigid coverslip. Cell adhesions, therefore, develop that are larger than they otherwise would be, which establishes just how far MSCs can feel: ˜1 μm on soft gels (<25 kPa). Accordingly, cells feel matrix strains localized to the scale of multiple adhesions, rather than the cellular scale.

Adhesions provide MSCs the necessary attachments to “feel” their microenvironment through acto-myosin contractions. Mechanically, contractility equates to a cellular pre-stress, σ, that balances the traction stresses, τ, exerted on the gel by the cell (Wang et al., Am. J Physiol. Cell Physiol. 282:C606-616 (2002b)). Traction stresses (τ, force per area) were determined from bead displacements in the gel (see FIG. 7B(inset)) by computing forces with available algorithms (Butler et al., Am. J. Physiol. Cell Physiol. 282:C595-605 (2002); Dembo et al., Biophys. J. 76:2307-2316 (1999); Schwartz et al., Phys. Rev. Lett. 88:048102 (2002); Wang et al., 2002, supra). Although larger tractions are exerted on stiffer gels, typical tractions of (τ)˜1 kPa exceed, by orders of magnitude, the viscous fluid drag on any cell crawling in culture. In addition, mean cell tractions equate to mean gel strains that differ very little (εₒᵤₜ=(τ/E)≅3 to 4%) between gels that differ by 2-fold in E. Consistent with nearly linear adhesion area increases with E, average σ for MSCs, C2C12-myoblasts, and hFOB-osteoblasts also show the same linear increase versus matrix stiffness, E (see FIG. 7B(top)). The trend implies larger deformation within the cell on stiffer matrices and larger deformation in the matrix on softer matrices.

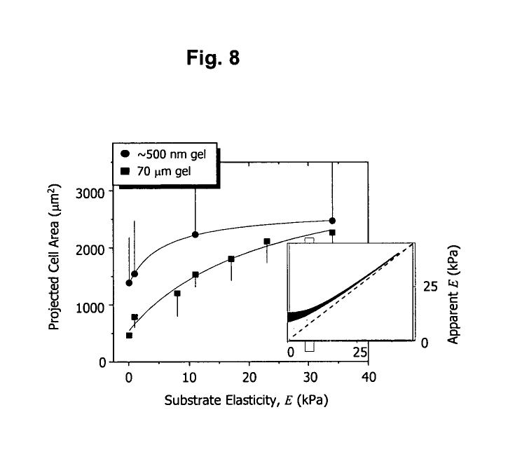

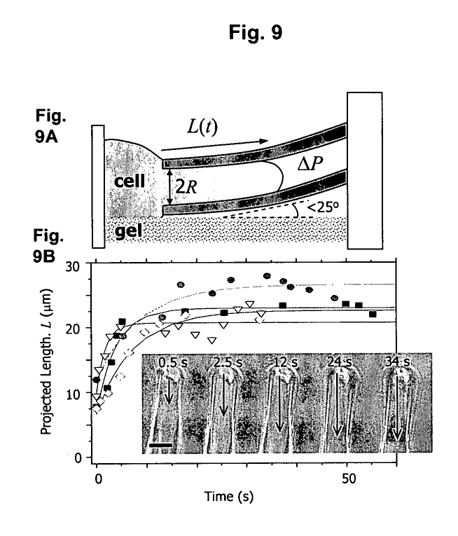

Blebbistatin, which was shown above to inhibit myosin contraction and expression, prevented any of the cells from developing either a pre-stress σ (Griffin et al., 2004, supra) or a significant cortical stiffness, κ, on any matrix (see FIG. 7B(bottom, open points)). The latter was measured by fitting a viscoelastic half-space model to membrane aspiration experiments (see FIG. 8 showing an example of cell spreading on an ultra-thin polyacrylamide gel). Cells contract their matrices up to 1-3 microns so that on thin, soft gels (h˜500 nm) attached to glass, cells are expected to ‘feel’ a matrix that is effectively stiffer than the cast gel. The result, as shown in FIG. 8, is an enhanced spreading of the cells, which allows mapping the spread area on thin gels, as compared with thick gels, and a determination of an ‘apparent’ gel modulus.

As functions of matrix stiffness, the two differentiated cell types, the C2C12-myoblasts and hFOB-osteoblasts exhibit similar (κ/E) slopes—though distinct intercepts. By using the pre-stress results, these two differentiated cell types also show the same slope for (κ/σ) (≈0.2) as highly contractile, smooth muscle cells assayed by different techniques (Wang et al., 2002b, supra). On the other hand, MSCs appear more mechano-sensitive, with twice the slope for (κ/E) and (κ/σ). This increased mechano-sensitivity leads to a self-consistent crossover. On myogenic gels (11 kPa), MSCs and C2C12s have similar κ, whereas on osteogenic gels (34 kPa), MSCs and hFOBs have similar κ. Despite this difference, the inside-outside relationship between intracellular strains, εᵢₙ (=σ/κ), and the extracellular strain field, εₒᵤₜ (=τ/E), fits a universal power law for all cell types (see FIG. 7B(inset)).

One can think of such a strain comparison as similar to comparing intracellular and extracellular ion concentrations (Na+, K+, Ca++, etc.). In the present invention, however, the inverse relationship between intracellular and extracellular strains reveals that, on stiff matrices, cell strains are large, while matrix strains are small. Whereas, by comparison, on soft matrices, cell strains are small, while matrix strains are large. The strain thus transfers from outside to in with increasing matrix stiffness, presumably activating different pathways at different strains. However, the common power law indicates a common mechanism, consistent with the central role of myosin II.

Effective Energetics for Lineage Specification

In an embodiment of the present invention, the effective energetics for lineage specification can be determined. For example, NMM II inhibition blocks lineage commitment (see FIG. 5B), but when cells are contractile, select activators/effectors appear to be choreographed to reprogram transcription as directed by matrix elasticity (see FIGS. 6A and 6B). Activator/effector species appear to be mobilized (FIG. 6A) by forces in cytoskeletally-linked adhesion structures (see FIGS. 6D and 6E) that grow with substrate stiffness (see FIG. 7B).

These findings are formalized in a simple model, wherein chemo-mechanical energetics localized to adhesions are balanced against the contractile energetics of the cell. Contractility or pre-stress, σ, is assumed to act throughout the cell volume V as a global regulator of differentiation. Coupled to this, an increase in free concentration of the local, transducing activator/effector links cooperatively to collagen (with Hill coefficient m and affinity K) and to substrate elasticity (E). The net result (see Example 2) is a lineage commitment probability given by:

$${P}_{lineage} ( E ) = {a}_{0} + {a}_{1} exp ( - σ V / {k}_{b} {T}_{eff} ) [ \frac{{E}^{m}}{{E}^{m} + {K}^{m} {coll}^{m}} ] \qquad Equation 2$$

The effective thermal energy k_{b}Teff in the exponential factor should relate more to cytoskeletal stochastics than to temperature. In the limit of rigid substrates where tensions (σ) are high, such as on glass, isometric pulling on adhesions will limit differentiation of MSC, as seen, e.g., in FIGS. 2C and 5B. See also Engler et al., supra, in press.

Equation 2 fits the three differentiation peaks of FIG. 2C (at E* ≈0.3 kPa, 10 kPa, 30 kPa) with best fit values for the key parameters K, m, and T_{eff} given in the legend. All of these parameters increase with increasing E*, and cooperativity, m, notably rises from about 2 to 8 (recall oxygen binds hemoglobin with m≈4), suggesting the progressive formation of large signaling complexes, consistent with growing adhesions (FIG. 6D).

The present invention is further described in the following examples. These examples are provided for purposes of illustration only, and are not intended to be limiting unless otherwise specified. The various scenarios are relevant for many practical situations, and are intended to be merely exemplary to those skilled in the art. These examples are not to be construed as limiting the scope of the appended claims. Thus, the invention should in no way be construed as being limited to the following examples, but rather, should be construed to encompass any and all variations which become evident in light of the teaching provided herein.

### EXAMPLES

### Example 1

The following Materials and Methods were utilized in the tunable systems used to provide exemplary proofs of the principles provided in the present invention.

Materials and Methods

Cell Culture: Human Mesenchymal Stem Cells (MSCS; Osiris Therapeutics; Baltimore, Md.), human osteoblasts (hFOBs; ATCC, Manassas, Va.), primary human skin fibroblasts (FC7) (Engler et al., 2004c, supra), and murine myoblasts (C2C12s; ATCC) were cultured in normal growth media listed in Table 1, supra. To chemically induce MSC differentiation, cells were placed in the appropriate induction media also listed in Table 1, supra. All cells were used at low passage number, and were subconfluently cultured. Cells were plated for experiments at ˜103 cells/cm³ and cultured for 7 days, unless otherwise noted. All chemicals were purchased from Sigma (St. Louis, Mo.) unless otherwise noted.

To inhibit proliferation, cells were exposed mitomycin C (10 μg/ml) for 2 hr and washed three times with media prior to plating. Blebbistatin (50 μM; EMD Biosciences, Inc., San Diego, Calif.), a NMM II inhibitor, was applied with every media change and was stable in culture media for up to 48 hours, as determined by thin layer chromatography.

Substrate Preparation: Cells were plated on variably compliant polyacrylamide gels according to a previously established protocol (Engler et al., 2004a, supra; Pelham et al., 1997, supra) herein incorporated by reference. Briefly, gel cross-linker N,N′methylene-bis-acrylamide and acrylamide monomer were varied in distilled water to achieve a polymerized solution with a tunable elastic modulus (Engler et al., 2004b, supra). Approximately 25 μl of the mixed solution was polymerized on a coverslip using 1/200 volume of 10% ammonium persulfate and 1/2000 volume of N,N,N′,N′-tetramethylethylenediamine. The polymerizing gel was covered with a dichlorodimethylsilane-pretreated coverslip to ensure easy detachment and a uniform polymerized gel surface. Final gels were 70-100-μm thick, as measured by microscopy.

To produce ultra-thin gels, however, 10 μl of a 1% polystyrene bead solution (d=250 nm; Polysciences, Inc., Warrington, Pa.) was added to polymerizing solutions, and a weight was added to the top coverslip to ensure that the gel thickness was defined by the spacer bead diameter. Type 1 collagen (0.25-1 μg/cm²; BD Biosciences, Rockville, Md.) was chemically cross-linked using a photoactivating cross-linker, sulfo-SANPAH (Pierce Biotechnology, Inc., Rockford, Ill.) and attachment was confirmed by fluorescence. Cells grown on glass coverslips (GL) alone were always coated non-specifically with collagen prior to cell seeding.

Differentiation Assays:

1) Morphological Changes and Immunofluorescence: Changes in cell shape (<4 days), especially the development of neurite-like branches (Engler et al., 2004c, supra) or spindle-like morphologies (Flanagan et al., 2002, supra), were quantified either by the number of membrane branches per mm of cell or by a “spindle factor,” referring to the major cell axis/minor cell axis, respectively. Cells also were stained with lineage-specific antibodies: myogenesis with Myogenesis Differentiation Protein 1 (MyoD1; Chemicon® International, Temecula, Calif.), osteogenesis with Core Binding Factor α1 (CBFα1 ; Alpha Diagnostic International, San Antonio, Tex.), and neurogenesis with phosphorylated and dephosphorylated Neurofilament Heavy chain (NFH; Stemberger Monoclonal, Berkeley, Calif.) along with paxillin (Chemicon), skeletal muscle myosin heavy chain (Zymed Laboratories, S. San Francisco, Calif.), and non-muscle myosin IIA and B (Sigma) or rhodamine-labeled phalloidin. Cells were fixed with formaldehyde, incubated in a 5% albumin blocking solution for 1 hour at 37° C., permeabilized with 0.5% Triton-X-100 and incubated overnight at 4° C. in 1:100 dilution of antibodies in PBS. Cells were then incubated for 1 hour at 37° C. in 1:500 FITC-conjugated secondary and 60 μg/mL TRITC-phalloidin. Finally, cells were incubated for 10 min. in 1:100 Hoechst 33342 (Molecular Probes Europe, Leiden, Netherlands) to label DNA. Cell morphology and fluorescently labeled cells were examined on a TE300 inverted epi-fluorescent Nikon or Olympus (TIRF) microscope, imaged on a cascade CCD camera (PhotoMetrics, Huntington, Beach, Calif.), and quantified with Scion Image (Scion Corp., Frederick, Md.).

Western blotting: Cells (with or without blebbistatin treatment, 50 μM) were also plated on 45×50 mm coverslips to obtain enough cells for western blotting. Cells were permeablized with lysis buffer (10% SDS, 25 mM NaCl, 10 nM pepstatin, and 10 nM leupeptin in distilled water), boiled for 10 minutes, placed in a reducing SDS-PAGE gel (Invitrogen, Carlsbad, Calif.) with MOPS buffer, and run against a colormetric molecular weight marker. Proteins were transferred onto nitrocellulose and blocked in a solution of 1% albumin, 50 mM Tris Buffered Saline (TBS). Membranes were rinsed 2X in 1 % Tween 20-TBS (TTBS), and then a 1:500 solution of primary antibodies were added for 2 hours. Membranes were rinsed 2X with TTBS again and 1:1000 of the secondary HRP-conjugated antibodies were added. Color development was achieved with a HRP development kit (Bio-Rad Laboratories, Hercules, Calif.). All westerns were run in duplicate, along with an addition blot for actin and Commassie blue-staining to ensure constant protein load among samples. Quantification of western blots was done by Scion Image software.

3) Oligonucleotide Array Assays: Total RNA (3-5 μg) was obtained from MSCs (with or without blebbistatin treatment) cultured on gel substrates of varying stiffness, as well as C2C12 myoblasts on 11 kPa gels and hFOB osteoblasts on 34 kPa gels, using an ethanol-spin column extraction. The samples were labeled with an Ampolabeling Linear Polymerase Reaction kit (SuperArray Bioscience, Frederick, Md.) and hybridized to custom oligonucleotide arrays. Membranes for control (C2C12, hFOB, and MSCs from flasks), experimental (MSCs on gels), and duplicate sample RNAs were processed in parallel to reduce technical variability. Chemiluminescent signals were detected on Biomax Film (Kodak) and analyzed with Scion Image software. Background-corrected signals were normalized to a control gene, β-actin.

Creep-test Micropipette Aspiration: Micropipettes were forged using a deFonbrune-type microforge (Vibratome, St. Louis, Mo.) to a radius of 2-3 μm with an approximately 25° pipette curvature so when mounted in micromanipulators (Nirishige; Japan) at an angle similar to the micropipette's curvature. The end of the pipette was flush with the cell edge (see FIG. 3A). A step pressure drop was imposed on the cell membrane, causing the membrane projection, L, to aspirate into the pipette as a function of time (t), pressure drop (ΔP) and pipette radius (R), as governed by the Sato and coworkers' viscoelastic half-space model (Sato et al., J. Biomech. Eng. 112:263-268 (1990), herein incorporated by reference:

$$L ( t ) = \frac{3 R Δ P}{πκ} [ 1 - \frac{{μ}^{′}}{κ + μ} exp ( \frac{- t}{τ} ) ] \qquad Equation 3$$

Images of the projection length were taken every 2 seconds in brightfield on a TE300 inverted Nikon microscope and cascade CCD camera (Photometrics), which allowed accurate fitting of the parameters ε, μ′, and τ to the data to determine the elastic and viscous moduli of each membrane.

Traction Force Measurements: Adhesive stresses, imposed on the matrix surface by an adherent cell, generate a displacement field from embedded beads within a soft substratum, which can be mapped on the cell if the gel can be approximated as a semi-infinite solid. Briefly, the well characterized traction force method (Butler et al., 2002, supra; Dembo et al., 1999, supra; Wang et al., 2002b, supra) uses bead displacements between images with, and without, the adherent cell to assemble a displacement field and determine Green's strain function given known material properties of the substratum (elastic modulus, poisson's ratio, etc). The traction field was used to obtain the cell pre-stress, i.e., the net tensile force over the cell-matrix interface carried by the actin cytoskeleton across a cross-sectional area of the cell that balances cell traction forces (Wang et al., 2002b, supra).

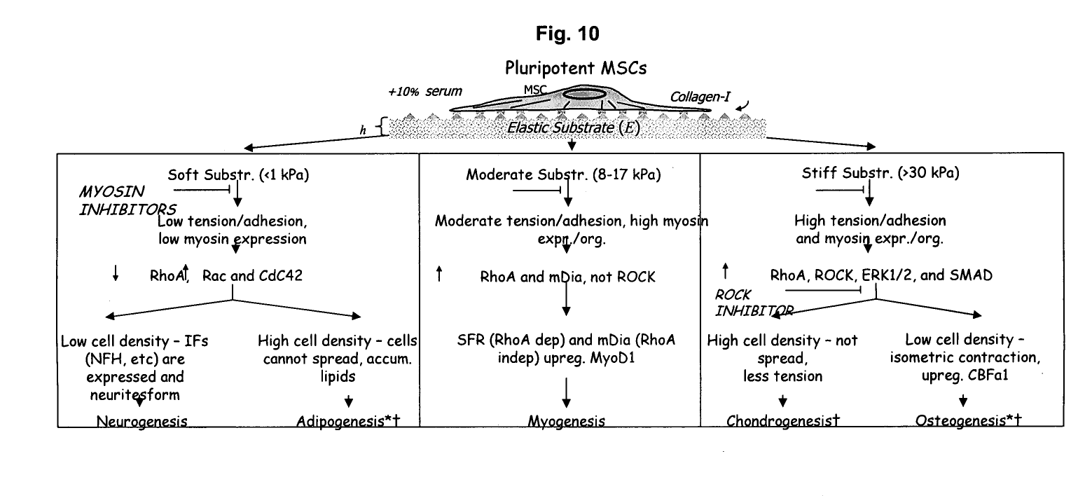

Accordingly, while stem cell differentiation by soluble stimuli is known, ihe strong influence of an immobilized microenvironment on cell differentiation is provided by the present invention. Myosins are known to have key roles in the relevant cell biology. NMM II, for example, is needed for neurite out-growth, as well as for myofibril assembly. RhoA associated shifts between mDia and ROCK have roles in cytoskeleton and mechano-sensing mechanisms via the serum response factor. Myosin signaling pathways in lineage commitment are summarized in FIG. 10, which illustrates elasticity-coupled lineage commitment. The microenvironment surrounding MSCs plays a critical role in lineage commitment by influencing cell adhesion and contractility via mechano-sensitive machinery, e.g., Rho GTPases. This reorganizes myosin to balance contractile forces against the sensed resistance which helps to localize and activate lineage-specific transcriptional programs.

### Example 2

Although the following model may be far too simplistic to capture the complexities of stiffness-controlled gene regulation, the model is inspired by the atomistic complexity of cooperative release of oxygen under hydrostatic tension (Carey et al., J. Biol. Chem. 252:4102-4107 (1977)). The goal of the following minimal model is thus to fit the differentiation results presented in FIG. 2C with the simplest formalism to incorporate the most essential physico-chemical ingredients. Two key states were assumed for the limiting association of a key, lineage-specific component, “Xᵢ.” This factor associates with apparent affinity K in or near the focal adhesions and obeys a molecular partition function (ξ), that cooperatively links to collagen (col) with a Hill coefficient (m) to give:
ξ=1+[(K/E) coll]^{m} Equation 4

In terms of energetics, K˜exp(−ΔG/k_{b}T) and the matrix modulus E˜A exp(εx²/k_{b}T). Additionally, ε is the relevant stiffness of matrix/membrane/adhesions, and x is a strain. If εx₂ is small, E˜A[1+(εx²/k_{b}T)] which implies that K is linear in E, as shown in FIG. 7B for cortical stiffness.

It was assumed that the fraction of unbound Xᵢ matters most for lineage specificity: θ′=1−[δIn(ξ)/In(coll)]=1/ξ. This is the free and diffusible fraction of Xᵢ (not associated with collagen) that has the strongest effect. With N as the total number of species Xᵢ, the total unbound portion of this species is Θ′=N θ′=N/ξ, which gives a chemo-mechanical potential for N=constant as:
G_{chem}=−k_{b}T In(N/ξ)=constant−k_{b}T In [1+[(K/E) coll]^{m}]⁻¹ Equation 5

The Total Free Energy depends additionally on the global pre-stress (σ), acting on the cell volume, V, Gₜₒₜ=G_{chem}+σV, which gives the lineage commitment probability (Equation5) by taking the exponential of Gₜₒₜ.

Mechano-biology is a broad field encompassing the recognition in the present invention that most tissue cells not only adhere to, but also pull on, their microenvironment, and as a result anchorage-dependent cells respond to the stiffness of the underlying matrix in ways that relate to tissue elasticity. In some aspects, microenvironments that are too soft or too stiff have implications in disease, as well as development, and highlight the need to understand the important role provided herein, for matrix physical properties and how cells feel the cellular matrix. For the cell biologist, the present invention offers methods that will lead to a better understanding of mechano-biological and/or mechano-chemical pathways and offer an understanding of more biologically relevant elastic substrates, as compared with rigid coverslips and polystyrene, for in vitro studies. For the applied biologist or bioengineer, the present invention will likely lead to modified strategies for tissue repair and cell scaffolding, such as the development of fibrous scaffolds for cell seeding, where careful attention can be given to fiber flexibility. Consequently, in addition to the regulation of differentiation of anchorage-dependent mesenchymal stem cells, many applications will result from the recognition that tissue cells feel and respond to the mechanics (elasticity) of their substrate.

All patents, patent applications and publications referred to in the present specification are also fully incorporated by reference.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the basic principles of the invention. Such modifications and additional embodiments are also intended to fall within the scope of the appended claims.

What is claimed is:

1. A method for regulating differentiation and cell shape of an anchorage-dependent cell, comprising:
selecting, designing, or engineering a substrate or tissue microenvironment having an elasticity defined by elastic constant E;
introducing the anchorage-dependent cell onto a substrate or into a microenvironment; and
developing the anchorage-dependent cell into a differentiated cell type, wherein shape and lineage commitment (in terms of gene or protein expression, or both) are regulated by the elasticity of the underlying substrate.

2. The method of claim 1, wherein the elasticity of the substrate ranges from 0.1 to 40 kPa.

3. The method of claim 2, wherein the elasticity of the substrate ranges from 0.1 to 1.0 kPa.

4. The method of claim 3, wherein the anchorage-dependent cell develops into a neurogenic-type cell.

5. The method of claim 2, wherein the elasticity of the substrate ranges from 8 to 17 kPa.

6. The method of claim 5, wherein the anchorage-dependent cell develops into a myogenic-type cell.

7. The method of claim 2, wherein the elasticity of the substrate ranges from 30 to 40 kPa.

8. The method of claim 7, wherein the anchorage-dependent cell develops into a osteogenic-type cell.

9. The method of claim 1, wherein the anchorage-dependent cell is a pluripotent mesenchymal stem cell.

10. The method of claim 1, wherein myosins and various markers for differentiation display stiffness sensitivity to the underlying substrate.

11. The method of claim 1, wherein the substrate comprises a tunable matrix.

12. The method of claim 11, wherein the substrate comprises an ultra-thin layer gel matrix and the method further comprises setting the elasticity of the substrate by selecting a concentration of cross-linking composition within the matrix, such that E is adjustable over several orders of magnitude, from extremely soft to stiff.

13. The method of claim 11, wherein the substrate comprises an adhesive ligand layer to which cells can bind.

14. The method of claim 11, wherein the substrate is a collagen I-coated polyacrylamide gel and the method further comprises setting the elasticity of the substrate by establishing a concentration of bisacrylamide cross-linking, whereby controlling the extent of polymer cross-linking in the gel permits E to be adjusted over several orders of magnitude, from extremely soft to stiff.

15. The method of claim 1, wherein the differentiated cell type is neurogenic, myogenic or osteogenic.

16. The method of claim 1, further comprising exposing the anchorage-dependent cell to an inhibiting agent to inhibit expression of a lineage-specific regulator.

17. A method for regulating differentiation and cell shape of an anchorage-dependent cell, said method comprising:
selecting, designing, or engineering a substrate or tissue microenvironment having an elasticity defined by elastic constant E;
introducing the anchorage-dependent cell onto the substrate; and
balancing chemo-mechanical energetics localized to cell adhesions against contractile energetics of the cell, σ, that balances cell traction stresses, τ, exerted by the cell on its underlying substrate, thereby controlling cell shape and lineage commitment.

18. The method of claim 17, wherein cell adhesions provide necessary attachments permitting the cell t6 feel its microenvironment, and adhesion area increases linearly with E, such that larger deformation within the cell occurs on stiffer matrices and larger deformation in the substrate occurs on softer matrices.

19. The method of claim 18, further regulating cell shape and differentiation by controlling cell strain, such that there is an inverse relationship between intracellular and extracellular strains so that on stiff matrices, cell strains are large, while matrix strains are small, and on soft matrices, cell strains are small, while matrix strains are large.
